(19) United States
(12) Patent Application Publication
(10) Pub. No.: US 2002/0169138 A1
(43) Pub. Date: Nov. 14, 2002

(54) Delivery vehicles for bioactive agents and uses thereof

(75) Inventors: Lawrence L. Kunz, Redmond, WA (US); Thomas R. Tice, Hoover, AL (US); Randell T. Libby, Redmond, WA (US); Christopher W. McDaniel, Hoover, AL (US)

Correspondence Address: NEEDLE & ROSENBERG P C, 127 PEACHTREE STREET N E, ATLANTA, GA, 30303-1811, US

(73) Assignee: Southern Research Institute

(21) Appl. No.: 10/073,744

(22) Filed: Feb. 11, 2002

Related U.S. Application Data
(63) Continuation of application No. 09/178,438, filed on Oct. 23, 1998, now abandoned.
(60) Provisional application No. 60/063,114, filed on Oct. 24, 1997.

(51) Int. Cl. A61K 48/00; A61K 31/704

(52) U.S. Cl. 514/44; 514/26

(57) ABSTRACT

Compositions for transporting a bioactive agent across a biological membrane include the bioactive agent, an oil, an oil-immiscible compound and a noncationic surface active agent. The compositions may deliver the bioactive agent through a chemical microporulation mechanism, which allows transfer of the agent across both cellular, intracellular organelle, and nuclear membranes. Compositions for nucleic acid delivery include nucleic acid, oil, oil-immiscible compound, noncationic surface active agent and essentially no cationic lipid, or include nucleic acid, oil, oil-immiscible compound and two noncationic surface active agents. The nucleic acid may be hydrophobically-modified, and be in combination with an oil, an oil-immiscible compound and at least one surface active agent. The compositions may be used for gene delivery to a cell, as well as delivery of other therapeutic agents.

### CROSS REFERENCE TO RELATED APPLICATIONS

This application is a continuation of application Ser. No. 09/178,438, filed Oct. 23, 1998, pending, which claims priority upon application Ser. Nos. 08/956,694 and 60/063,114, both filed Oct. 24, 1997, both abandoned, which applications are hereby incorporated by this reference in their entireties.

### TECHNICAL FIELD

The present invention is generally related to therapeutic compositions, and more specifically to delivery vehicles in combination with a gene or other therapeutic agent.

### BACKGROUND OF THE INVENTION

Gene therapy is a relatively new approach for the treatment of inherited and acquired diseases. Therapeutic treatment generally requires a delivery system to transfer a gene sequence(s) into the target cell in order to achieve clinical benefits for the recipient. At present, two major strategies have been employed to accomplish gene delivery, one uses a virus as a vector and the other uses a non-viral vector as a delivery vehicle. Significant deficiencies currently exist with the former method of gene delivery.

A major disadvantage of the viral-mediated delivery system relates to the concern about its safety with respect to the possibility of recombination with endogenous virus resulting in a deleterious infectious form of the virus. Additionally, the use of a viral-based delivery system has been known to induce an immune response against the intrinsic viral antigens, rendering repetitive treatments with the same delivery vehicle problematic. As an example, the use of adenovirus vectors during gene delivery have been demonstrated to be slightly “leaky” in that they express low levels of adenovirus genes, causing the target cell to be identified by viral-specific T-lymphocytes with subsequent inflammation and target cell destruction. See Caplen, N.J. et al., Nature Med. 1:39-46, 1995. While viral-based vectors such as adenovirus (serotypes 2 and 5) can be an efficient gene transfer vehicle, there exists a size limit of foreign DNA which can be packaged within the viral genome. Moreover, adenoviral vectors are known not to be capable of transferring genes to non-dividing cells. See Miller D. G. et al., Mol. Cell Biol. 10:4239-4242, 1990. More recently, the use of viral-based genetic delivery vectors has been demonstrated to cause random integration of genes into the host chromosome. See, e.g., Temin, H. M., Human Gene Therapy 1:111-123, 1990. As a result of the inflammation/immunity issues associated with the use of viral vectors, many investigators have focused on devising alternative gene transfer systems.

Recently, non-viral delivery systems have drawn increasing attention in the field of gene therapy as a result of their relative simplicity and non-immunogenicity, as well as apparent low toxicity. The most commonly used formulation for non-viral gene delivery involves the use of cationic liposomes in which the negatively charged nucleic acid molecule is held within the positively charged liposome to make a complex. Although this form of non-viral gene delivery has been demonstrated in cultured cells and animals, severe drawbacks preclude its widespread use as a delivery vehicle. One of the major drawbacks of liposomal formulations is that the DNA/liposome complexes are not stable because they form aggregates and flocculates, which reduces the efficiency of in vivo gene transfer. Thus, it would be necessary to prepare complexes freshly and use them within a short period of time.

Additionally, it is known that the transfection activity of cationic liposomes is interfered with by serum components which presumably neutralize the unpaired positive charges in the complexes which are essential for binding of DNA/lipid complexes to the cell surface. See, e.g., Felgner, P. L. et al. Nature 337:387-388, 1989; Felgner, P. L., et al. Proc. Natl. Acad. Sci. USA 84:7413-7417, 1987; and Gao, X. and Huang, L., Biochem. Biophys. Res. Comm. 179:280-285, 1991. Because of this problem alone, the use of cationic liposomes for gene transfer is limited to the situation where a minimal amount of serum is present; accordingly, cationic liposomes as an in vivo delivery system are of limited value.

The need for safe and effective delivery vehicles for gene therapy is widely cited. See, e.g., Vera, I. M. and Somia, N. Nature 389:239-242, 1997 and Scientific American Special Report: Making Gene Therapy Work, pp. 96-106, June 1997. The present invention is directed to meeting this need and provides additional related advantages as disclosed herein.

### SUMMARY OF THE INVENTION

The present invention is directed to delivery vehicles for, and containing, bioactive agents, as well as methods associated therewith. One or more bioactive agents, or modified bioactive agent(s), may be added to a delivery vehicle of the present invention, and the combination used to delivery the bioactive agent(s) to, and then into, cells.

In one embodiment, the present invention provides a composition for transporting a bioactive agent across a biological barrier. The composition includes a bioactive agent, an oil, an oil-immiscible compound and a noncationic surface active agent. The composition is able to transport the agent across the barrier.

In another embodiment, the invention provides a composition that includes a nucleic acid, an oil, an oil-immiscible compound, a noncationic surface active agent, and essentially no cationic lipid.

In another embodiment, the invention provides a composition that includes a nucleic acid, an oil, an oil-immiscible compound, and two noncationic surface active agents.

In another embodiment, the invention provides a composition that includes a hydrophobically-modified nucleic acid, an oil, an oil-immiscible compound and at least one surface active agent.

In another embodiment, the invention provides a method of forming a composition that includes a bioactive agent. The method includes:

(a) combining the bioactive agent, an oil component, an oil-immiscible component, and at least two noncationic surfactants; and

(b) mixing the combination of step (a) to form the composition.

In another embodiment, the invention provides a method of forming a composition that includes a bioactive agent. In this embodiment, the method includes:

(a) combining the bioactive agent, an oil component, an oil-immiscible component, and a noncationic surface active agent in the substantial absence of a cationic lipid; and

(b) mixing the combination of step (a) to form the delivery composition.

In another embodiment, the invention provides a method of transporting a bioactive agent across a biological barrier. The method includes:

(a) contacting a composition comprising the bioactive agent with a biological barrier; and

(b) transferring the bioactive agent across the barrier by chemical microporulation.

In another embodiment, the invention provides a method of treating a plant or animal subject in need thereof with a therapeutic or prophylactic bioactive agent. The method includes administering at least one of composition (a), (b) or (c) to the subject:

(a) a composition including a nucleic acid as a bioactive agent, an oil, an oil-immiscible compound, a noncationic surface active agent, and essentially no cationic lipid;

(b) a composition including a nucleic acid as a bioactive agent, an oil, an oil-immiscible compound, and two noncationic surface active agents; and

(c) a composition including a hydrophobically-modified nucleic acid as a bioactive agent, an oil, an oil-immiscible compound and at least one surface active agent.

In another embodiment, the invention provides a method of preparing a composition for transporting a bioactive agent across a biological membrane. The method includes:

(a) modifying a nucleic acid to provide the bioactive agent, the bioactive agent being more hydrophobic than the nucleic acid; and

(b) combining the bioactive agent with a composition selected from

(i) an oil, an oil-immiscible compound, a noncationic surface active agent, and essentially no cationic lipid; and

(ii) an oil, an oil-immiscible compound, and two noncationic surface active agent.

In another embodiment, the compositions of the present invention are provided for use as a medicament, and for use for the manufacture of a medicament to provide a therapeutic or prophylactic bioactive agent to a subject. The present invention further provides for a method comprising the administration, to a patient in need of one or more bioactive agents, of a composition containing one or more bioactive agents and a delivery vehicle according to the present invention.

These and other embodiments of the present invention will become evident upon reference to the following detailed description and drawings.

### BRIEF DESCRIPTION OF THE DRAWINGS

FIG. 1 is a schematic illustration of direct and indirect in situ polymerase chain reaction.

FIG. 2 illustrates the effect on de novo DNA synthesis in two cell lines of contact with specific microemulsions.

FIG. 3 illustrates the transfection ability in two cell lines of W/O emulsions of the invention containing digoxigenin-labeled DNA.

FIG. 4 illustrates that internalization of a transfected digoxigenin-lacZ fragment from both W/O and O/W emulsions can occur.

FIGS. 5-13 are electron micrographs illustrating microporulation with three different compositions of the invention.

FIG. 14 illustrates that the lacZ gene has inserted into rabbit arterial tissue as detected by a 520 basepair product from the polymerase chain reaction.

FIG. 15 is an electron micrograph image of a condensed lacZ gene construct (50,000×).

FIG. 16. is a computer enhanced and background extracted electron micrograph image of a condensed lacZ gene construct from FIG. 15.

FIG. 17 illustrates a pseudo-ternary phase diagram for surfactant (which includes multiple surfactants/co-surfactants)-water-oil mixtures.

### DETAILED DESCRIPTION OF THE INVENTION

As stated briefly above, the present invention provides delivery vehicles for, and/or containing, bioactive agents, as well as methods associated therewith. Thus, one or more bioactive agents (which term includes modified bioactive agents), may be added to a delivery vehicle of the present invention, and the combination used to delivery the bioactive agent(s) to, and then into, cells. The method may be used in either an ex vivo or in vivo manner to deliver bioactive agents to one, or preferably many, cells.

In one embodiment, the bioactive agent is nucleic acid, preferably nucleic acid useful in gene therapy. Nucleic acid, and cell surfaces, are both negatively charged, and accordingly much use has been made to date of positively charged lipids to prepare emulsions and micelles for the delivery nucleic acid across cell membranes and into cells. See, e.g., PCT International Publication No. WO 97/11682. One problem with this approach is that the delivery vehicle typically does not pass through the membrane. Accordingly, when the nucleic acid is delivered inside the cell, it is exposed to lysosomes and other degradative conditions, which can degrade the nucleic acid and reduce its expression.

One feature of the present invention is the recognition that cationic lipid is not necessarily present, or even desirable, in an emulsion or micellar delivery vehicle. In the absence of cationic lipid, nucleic acid-containing droplets have been observed to pass through a cell membrane in a manner that allows the integrity of the delivery vehicle to be retained.

The present invention generally provides compositions that contain, or are prepared from, delivery vehicle and bioactive agent(s), where the delivery vehicle contains, or is prepared from, components including an oil, an oil-immiscible compound, and specific surface active agent(s). These compositions may also be referred to as pharmaceutical compositions. Each of the components is described below.

As used herein, an “oil” refers to a non-charged chemical that is either completely or substantially insoluble in water. The oil may also be referred to as a lipophilic compound, an oleophilic compound, or a hydrophobic compound. The oil is preferably a liquid at room temperature. A preferred oil is physiologically tolerable by the host into which the oil is introduced (i.e., clinically acceptable). Preferably, the oil is miscible with water to an extent of less than 0.1 g oil per 100 g water at 25° C.

As discussed in the art, oils are commonly classified by their origin. According to this classification, suitable oils of the invention may be from any origin, including but not limited to animal, vegetable, mineral, and synthetic origin.

Suitable oils of animal origin include, without limitation, those oils which are found in tallow and lard, as well as liquid animal types including fish oil, cod-liver oil, halibut, shark liver and sperm whale oil.

Suitable oils of vegetable origin include, without limitation, oils derived primarily from seeds or nuts, and include castor, coconut, corn, cottonseed, ethereal, linseed, olive, palm, peanut, rapeseed, safflower, sesame, and soybean oils. The vegetable oil may be a so-called essential oil, which is a term commonly used to refer to mixtures of water-immiscible materials obtained from extraction of vegetation such as flowers, stems, leaves, or even the entire plant. These essential oils include, without limitation, terpenes such as one or more pinene isomers and dipentene (limonene).

Suitable oils of mineral origin may be derived from petroleum and include, without limitation, aliphatic or wax-based hydrocarbons, aromatic or asphalt-based hydrocarbons or mixed aliphatic and aromatic based hydrocarbons. Also included in the mineral classification are petroleum-derived oils such as refined paraffin oil, and the like. The term “mineral oil” also refers to a highly refined, colorless, tasteless, and odorless petroleum oil (i.e., derived by processing petroleum/crude oil) used medicinally as an internal lubricant and for the manufacture of salves and ointments. Such mineral oils are highly refined in having substantially all volatile hydrocarbons removed therefrom, and in being hydrogenated (also called hydrotreated) in order to remove substantially all unsaturation, e.g., aromatic groups have been reduced to the fully saturated analog. A preferred mineral oil to prepare a composition of the invention is so-called “white” mineral oil, which is water-white (i.e., colorless and transparent) and is generally recognized as safe for contact with human skin. Mineral oil may also be characterized in terms of its viscosity, where light mineral oil is relatively less viscous than heavy mineral oil, and these terms are defined more specifically in the U.S. Pharmacopoeia, 22^{nd} revision, p. 899 (1990).

Suitable oils of synthetic origin include, without limitation, mono-, di- and tri-glycerides of saturated and unsaturated (C₆-C₂₂) fatty acids, as well as their ethoxylated derivatives. The same oils may be naturally occurring and be equally suitable for use in the invention. The oil may be a silicon oil.

Other suitable synthetic oils are so-called “fluorochemicals”, where this term refers to fully, highly, or partially fluorinated organic compounds. The fully fluorinated compounds may be referred to as perfluorocompounds (PFC), which have fluorine atoms, instead of hydrogen atoms, bonded to the carbon backbone. In general, fluorochemicals are commercially available or may be synthesized by well-known chemical or electrochemical processes. The term “fluorochemical” includes cyclic as well as acyclic compound of carbon. Substituted derivatives thereof are also included, so that fluorochemicals may include atoms other than fluorine and carbon, such as hydrogen, oxygen, nitrogen and bromine, etc. Among the perfluorinated organic compounds which may be employed as an oil are perfluorotributylamine, perfluorodecalin, perfluoromethyldecalin, perfluorooctylbromide, perfluorotetrahydrofuran, and perfluoroethers including (CF₃)₂CFOCF₂(CF₂)₂CF₂OCF(CF₃)₂, (CF₃)₂CFOCF₂(CF₂)₆CF₂OCF(CF₃)₂, perflourododecane, perfluorobicyclo[4.3.0]nonane, perfluorotrimethylcyclohexane, perfluorotripropylamine, perfluoroisopropylcyclohexane, perfluoroendotetrahydrodicyclopentadiene, perfluoroadamantane, perfluoroexo-tetrahydrodicyclopentadiene, perfluorobicyclo[5.3.0.]decane, perfluorotetramethyl-cyclohexane, perfluoro-1-methyl-4-isopropylcyclohexane, perfluoron-butylcyclo-hexane, perfluorodimethylbicyclo[3.3.1.]-nonane, perfluoro-1-methyl adamantane, perfluoro-1-methyl-4-t-butylcyclohexane, perfluorodecahydroacenaphthene, perfluoro-trimethylbicyclo[3.3.1.]nonane, perfluoro-n-undecane, perfluorotetradecahydro-phenanthrene, perfluoro-1,3,5,7-tetramethyladamantane, perfluorododecahydrofluorene, perfluoro-1,3-dimethyl adamantane, perfluoro-n-octylcyclohexane, perfluoro-7-methyl bicyclo[4.3.0.] nonane, perfluoro-p-diisopropylcyclohexane, and perfluoro-m-di-isopropylcyclohexane. Chlorinated perfluorocarbons, such as chloroadamantane and chloromethyladamantane as described in U.S. Pat. No. 4,686,024 may also be used. Such compounds are described, for example, in U.S. Patent Nos. 3,962,439; 3,493,581, 4,110,474, 4,186,253; 4,187,252; 4,252,827; 4,423,077; 4,443,480; 4,534,978 and 4,542,147, European Pat. Applns. Nos. 80710 and 158,996, British Pat. Specification 1,549,038 and German Offen. 2,650,586. Of course, it should be understood that mixtures of any of these highly fluorinated organic compounds may also be used in the emulsions and processes of this invention.

Another classification for oil commonly employed in the art refers to the chemical composition of the oil. According to this classification, suitable oils of the invention may have any chemical structure providing oil properties, including, but not limited to, hydrocarbons and compounds having one or more of halogen, ester, ether, hydroxyl, amide, substitution on a carbon backbone. Suitable oils may have hydroxyl substitution on a carbon backbone, e.g., may be monohydric or polyhydric aliphatic alcohols, such as hexadecyl alcohol, 2-octyldodecanol, oleyl alcohol, etc. Alkoxylated derivatives thereof may also be suitable. Lecithin may be used as the oil component.

Another group of suitable oils of the present invention are sometimes referred to in the art as liquid waxes, where this term has been used to refer to esters, such as isopropyl myristate, caprinate, caprylate, laurate, palmitate and stearate; oleates, e.g., olcyl oleate, ethyl oleate, etc. The oil may be a monoester having at least 8 carbon atoms and a formula (C₁-C₃₆)—C═O—O—(C₁-C₃₆), or a diester having at least 8 carbon atoms and a formula (C₁-C₃₆)—C═O—O—(C₂-C₃₆)—C═O—O—(C₁-C₃₆). The oil may be in combination with one or more organic chemicals having less than 8 carbon atoms.

As used herein, an “oil-immiscible compound” refers to a compound which is not, or is only slightly miscible with the oil as defined herein and as selected for a particular composition. Water and polyhydric organic molecules are exemplary and non-limiting illustrations of oil-immiscible compounds. The oil-immiscible compound is preferably a physiologically-acceptable aqueous phase, such as physiological saline or aqueous phosphate buffer, selected in accordance with the route of administration and standard pharmaceutical practice. Generally, normal saline is a suitable pharmaceutically acceptable aqueous phase. Other suitable aqueous phases include, e.g., water, buffered water, 0.4% aqueous saline, 0.3% aqueous glycine, aqueous solutions containing sugar such as lactose, dextrose, and the like, The oil-immiscible compound may include a water-miscible organic alcohol, such as methanol, ethanol, propanol, ethylene glycol, propylene glycol, and glycerol. Preferably, the oil-immiscible compound is soluble in corn oil to an extent of less than 0.1 g oil-immiscible compound per 100 g corn oil at 25° C. Water, preferably without an organic cosolvent, is a preferred oil-immiscible compound. Accordingly, aqueous compositions, preferably without an organic cosolvent, are suitably employed to provide the oil-immiscible compound in the delivery vehicles and compositions of the invention.

As used herein, a “surface active agent” refers to a chemical that has both hydrophilic and hydrophobic portions. A surface active agent can be characterized by a “hydrophile-lipophile balance number” (HLB). The HLB is a characteristic of individual surface active agents that can be either calculated or determined empirically, as previously described (see, e.g., Schick, M. J., Nonionic Surfactants, p. 607 (NY: Marcel Dekker, Inc. (1967)). HLB can be calculated by the following formula: 20×MW hydrophilic component/(MW hydrophobic component+MW hydrophilic component), where MW=molecular weight (see, e.g., Rosen, M. J., Surfactants and Interfacial Phenomena, pp. 242-245, John Wiley, New York (1978)). The HLB is a direct expression of the hydrophilic character of the surface active agent, i.e., the larger the HLB, the more hydrophilic the compound. The surface active agents useful in the present invention may also be characterized in terms of their phase inversion temperature, which is also known in the art as a PIT value.

Typically, an oil-soluble surface active agent will have an HLB value less than 8. A typical water-soluble surface active agent will have an HLB value greater than 10. Depending on the need and objective, one skilled in the art is able to choose an existing surface active agent, or to combine surface active agents, to achieve a desired HLB value. The surface active agents can be chosen from existing compounds, or may be synthesized.

The surface active agent may be a noncationic surface active agent. As used herein, a “noncationic surface active agent” refers to a surface active agent that does not carry a net positive charge. The noncationic surface active agent may either have a net neutral charge or a net negative charge. In one embodiment, the noncationic surface active agent does not contain any positive charge, while in another embodiment, the noncationic surface active agent is negatively charged (and does not have any positive charges, i.e., is not zwitterionic). A preferred noncationic surface active agent is a nonionic surface active agent which does not bear any positive or negative charges. In one embodiment, the nonionic surface active agent contains a hydrophobic organic group in covalent attachment to a hydrophilic polyol. In general, the hydrophobic organic group can be, for example, an alkyl chain, an aralkyl group, an aryl group, or an alkylaryl group. An alkyl chain can be chosen of any desired size, depending on the hydrophobicity desired and the hydrophilicity of the polyol moiety. A preferred range of alkyl chains is from 4 to 24 carbon atoms. An aryl group can consist of, e.g., a phenyl group, a naphthyl group, an anthracene group, a phenlanthrene group, or a flavonoid group. An aralkyl group can consist of, e.g., a benzyl group, a tolyl group, a p-isooctylphenyl group, a 2-methylazulene group or a methylumbelliferyl group. A steroid group can be chosen from, for example, sapogenin estradiol, cholesterol or cortisol. In general, the hydrophilic polyol can be, for example, a monosaccharide, a disaccharide, an oligosacchiaride, a sugar alcohol, a polyoxyethylene, a polyoxypropylene, a copolymer of polyoxyethylene and polyoxypropylene (sometimes referred to as poloxamers in the art), or a polyoxyethylene sorbitan.

Suitable nonionic surface active agents for the present invention are alkyl glycosides. Suitable alkyl glycosides can be synthesized by known procedures, i.e., chemically, as described, e.g., in Rosevear et al., Biochemistry 19:4108-4115 (1980) or Koeltzow and Urfer, J. Am. Oil Chem. Soc. 61:1651-1655 (1984), U.S. Pat. Nos. 3,219,656 and 3,839,318, or enzymatically, as described, e.g., in Li et al., J. Biol. Chem. 266:10723-10726 (1991) or Gopalan et al., J. Biol. Chem. 267:9629-9638 (1992).

Another suitable nonionic surface active agent is polyoxyethylene joined with an organic grouping. Such agents include, without limitation, nonaethylene glycol octylphenyl ether, e.g., Triton X-100™ (Rohm and Haas, Philadelphia, Pa.) or NP-40™ (Pierce Chemical Co., Rockford, Ill.), heptaethylene glycol octylphenyl ether, e.g., Triton X-114™ (Pierce Chemical Co.) and others (see, e.g., Enyeart, C. R., “Polyoxyethylene Alkylphenols” in Nonionic Surfactants, M. J. Schick, ed., 44-85 (Marcel Dekker, NY, 1967). Synthesis of desired polyoxyethylene compounds can be achieved as described in, e.g., Fine, R. D., J. Am. Oil Chem. Soc. 35:542 (1958).

Other suitable nonionic surface active agents are alkyl polyoxyethylene sorbitans, such as polyoxyethylene sorbitan monolaurate (Tween-20™), polyoxyethylene sorbitan monopalmitate (Tween-40™), polyoxyethylene sorbitan monooleate (Tween-80™) (Bio-Rad, Richmond, Calif.; Calbiochem, San Diego, Calif.), and others (see, e.g., Benson, F. R., Polyol Surfactants in Nonionic Surfactants, pp. 247-299); alkyl sorbitans, such as sorbitan monolaurate (Span 20™), sorbital monopalmitate (Span 40υ, sorbital monostearate (Span 60™), and sorbital monooleate (Span 80™) (Sigma, St. Louis, Mo.); and polyoxyethylene stearyl ethers, such as Brij 72™, Brij 76™, Brij 78™, and Brij 100™ (Sigma, St. Louis, Mo.).

Another suitable nonionic surface active agent is a maltose polyol linked by glycosidic linkage to an alkyl chain of 12, 13 or 14 carbon atoms, e.g., dodecyl maltoside, tridecyl maltoside and tetradecyl maltoside. These compositions are commercially available (Anatrace, Inc.) and are nontoxic (see Weber and Benning, J. Nutr. 14:247-254 (1984), reporting that orally administered alkyl glycosides are metabolized to nontoxic metabolites).

The above examples are illustrative of the types of surfactants to be used in the compositions and methods claimed herein; however, the list is not exhaustive. Derivatives of the above compounds which fit the criteria of the claims should also be considered when choosing a surfactant. All of the compounds can be screened for efficacy following the methods taught in the Examples set forth herein.

As used herein, the term “bioactive agent” refers to a molecule, compound or complex that is advantageously introduced into a biological host. The bioactive agent is also referred to in the art as a physiologically or pharmaceutically active compound; a biologically active compound, agent or substance; and active, therapeutic, or prophylactic agent. The bioactive agent, when present in an effective amount, reacts with and/or affects living cells and organisms. The bioactive agent may exert its influence through action on the cell surface and/or exert its influence through activity within the cell (including acting upon or within an intracellular organelle), but preferably exerts its influence within the cell.

The bioactive agent may be a drug. The term “drug” as used herein encompasses the “drugs” defined by the Federal Food, Drug and Cosmetic Act, which are articles recognized in the official United States Pharmacopoeaia, official Homeopathic Pharmacopoeaia, or official National Formulary or any supplement to any of them. The term “drug” as used herein also encompasses the definition of the same term as found in Remington's Pharmaceutical Sciences, which is any article contained in the official United States Pharmacopoeaia, official Homeopathic Pharmacopoeaia, or official National Formulary or any supplement to any of them, which is used in the process of diagnosis, cure, treatment, mitigation or prevention of disease in man or animals (see Remington's Pharmaceutical Sciences, Easton, Pa., Mack Publishing Co., 1975, p. 1843). For purposes of the present invention, the term “drug” as used herein additionally encompasses any article which may be used in the process of diagnosis, cure, treatment, mitigation or prevention of disease in a plant. Water is not a drug or a bioactive agent.

The bioactive agent may be a vaccine. For a discussion of vaccines suitable for use as a bioactive agent in compositions of the present invention, see, e.g., Shearer, G. M. et al. TIBTECH 15:106-109 (March 1997); Robinson, H. L. Vaccine 15(8):785-787 (1997); Donnelly, J. J. et al. Life Sciences 60(3):163-172 (1997); and Donnelly J. J. et al. Annu. Rev. Immunol. 15:617-648 (1997). See also the following references, which are relevant to cancer vaccines: Wolff, J. A, et al., Science 247:1465-1468, 1990; Donnelly, J. J., et al., Annual Rev, Immunol. 15:617-648, 1997; Lee, R. J., et al., Critical Reviews in Therapeutic Drug Carrier System 14:173-206, 1997; and Rosenfield, M. E., et al., Current Opinion in Oncology 8:72-77, 1996.

Exemplary bioactive agents of the present invention include, without limitation, an adrenergic receptor antagonist such as propanolol; an anesthetic such as dibucaine or procaine; an anti-arrhythmic, an anti-bacterial compound such as carbenicillin, gentamycin, mexiocillin, or penicillin G; an anti-coagulant such as heparin; an anti-depressant such as desipramine; an anti-fungal compound such as amphotericin B; an anti-glaucomic agent such as pilocarpine or timolol; an anti-inflammatory compound such as aurothioglucose, dexamethasone, or gold sodium thiomalate; an anti-neoplastic compound such as azauridine, bleomycin, cisplatin, cytarabine, doxorubicin, fluorouracil, methotrextate, mitomycin C, neomycin, thioguanine, vinblastine, or vincristine; an anti-parasitic compound such as antimony complexes, mebendazole, or suramin; an anti-rheumatic; an anti-viral compound such as chloroquine or a rifamycin; a cell receptor binding molecule such as estrogen receptor protein; a drug; a dye such as Arsenazo III; an enzyme such as α-amylase, alkaline phosphatase, catalase, glucagon, horseradish peroxidase, lipase, tyrosine hydroxylase, caspase, or superoxide dismutase; a fluorescent compound such as carboxy fluoroscein; a gene such as the bcl-2 cell suicide suppresser gene or the nerve growth factor gene; a glycoprotein such as hyaluronic acid; a hormone such as estrogens; an immunodialator such as an interferon or interleukin; an immunoglobulin such as IgG or IgA; a lipoprotein such as α-lipoprotein; a neurotransmitter such as acetylcholine; a non-steroidal anti-inflammatory compound such as ibuprofen, indomethacin, naproxen, piroxicam, salicylic acid acetate, or sulindae; a nucleic acid or analog thereof such as adenine, guanine, cytosine, thymine or uracil; a polynucleic acid or analog thereof such as ssDNA, dsDNA, cDNA, plasmid DNA, RNA, mRNA, ribozyme, decoy RNA, peptide bonded nucleic acid (PNA), or antisense polynucleic acid; a peptide such as atrial natriuetic factors, bombesin antagonists, bradykinin antagonists, calcitoninis, chlorecystokininis, cyclosporin A's, cytoprotective peptide-cyclolinopeptides, desmospressin, elcatonin, endothelins, enkephalins, gastrin antagonists, gastrin releasing peptides, growth hormone releasing peptide, hirudins, insulins, interleukins, MSH-releasing factors, neurotensin antagonists, oxytocin agonists or antagonists, peptidergic antineoplastic agents, RGD peptides, somatostatins, or vasopressin agonists or antagonists; a polysaccharide such as glycogen; a protein such as albumin, cytokines, hemoglobin, Bcl-2 family members (including subfamilies Bcl-2, Bax and BH3) or interleukins; a prostaglandin; a radioactive compound; a toxin such as diphtheria toxin; a vaccine; a vitamin such as vitamin B complex, vitamin A, vitamin, D, vitamin E, vitamin K, vitamin B12 or folic acid; an agent bonded to a targeting molecule, where exemplary targeting molecules include, without limitation, an antibody and a fragment thereof, e.g., a Fab. The bioactive agent may be a marker, e.g., a dye, fluorescent molecule, or an enzyme that reacts with a chromogenic substrate.

A preferred bioactive agent is a nucleic acid. As used herein, “nucleic acid” or “nucleic acid molecule” refers to any of deoxyribonucleic acid (DNA), ribonucleic acid (RNA), oligonucleotides, fragments generated by the polymerase chain reaction (PCR), and fragments generated by any of ligation, scission, shearing, endonuclease action, and exonuclease action. Nucleic acids can be composed of monomers that are naturally-occurring nucleotides (such as deoxyribonucleotides and ribonucleotides), or analogs of naturally-occurring nucleotides (e.g., α-enantiomeric forms of naturally-occurring nucleotides), or a combination thereof. The nucleic acids suitable for use in the present invention will also include those nucleic acids in which modifications have been made in one or more sugar moieties and/or in one or more of the pyrimidine or purine bases. Examples of sugar modifications include replacement of one or more hydroxyl groups with halogens, alkyl groups, amines, azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purifies or pyrimidines, or other well-known heterocyclic substitutes. Nucleic acid monomers can be linked by phosphodiester bonds or analogs of such linkages, including linkages that are non-polar (lipophilic). Analogs of phosphiodiester linkages include phosphorothioate, phosphorodithioate, phosphoroselenoate, phosphorodiselenoate, phosphoroanilothioate, phosphoranilidate, phosphoramidate, and the like. The term “nucleic acid” also includes so-called “peptide nucleic acids,” which comprise naturally-occurring or modified nucleic acid bases attached to a polyamide backbone.

The nucleic acids can be either single-stranded or double-stranded molecules. The nucleic acids can be single-stranded DNA or RNA, double-stranded DNA, or DNA-RNA hybrids. Examples of double-stranded DNA include structural genes, genes including regulatory and transcriptional initiation and termination regions, and self-replicating systems such as plasmid DNA. Examples of other single-stranded nucleic acids include anti-sense oligonucleotides (complementary to DNA and RNA), ribozymes and triplex-forming oligonucleotides. Single-stranded nucleic acids can be susceptible to degradation, therefore, some single-stranded nucleic acids will preferably have some or all of the nucleotide linkages substituted with stable, non-phosphodiester linkages, including, for example, phosphorothioate, phosphorodithioate, phosphoroselenate, or O-alkyl phosphiotriester linkages.

The nucleic acids can be linear or circular molecules. The linear nucleic acids can be ligated together to form concatemers. The linear nucleic acids can have covalently closed ends. The circular nucleotide polymer will typically comprise either relaxed, nicked circular, or supercoiled, or a mixture thereof. Optionally, where linear polymers are preferred, circular molecules may be linearized using, for example, restriction enzymes as described in the art. The nucleic acids which are useful in the present invention (including both the complexes and droplets) are typically nucleotide polymers having from about 10 to about 200,000 nucleotide residues. The nucleotide polymers are physically incorporated into the droplets to be delivered as a structural component of the microemulsion. Accordingly, when the nucleotide polymer has greater than about 10,000 nucleotide residues, and preferably when the polymer has greater than about 100,000 nucleotide residues, it may be desireable to condense the polymer surface.

To the extent that a specific nucleic acid for is preferred, condensation methodology, also known as compaction, compression, or supercoiling, is known in the art and such methodology may be applied in the present invention to provide a condensed nucleic acid. In one embodiment, a condensed nucleic acid is the bioactive agent, as used herein. A condensed nucleic acid may serve as a bioactive agent in any of the compositions and delivery vehicles of the present invention. The condensed nucleic acid will typically become complexed with cationic species, and thus will become hydrophobically-modified as well as condensed. In a preferred embodiment of the invention a functional nucleic acid will be condensed to a size where each fragment will physically fit within the lipid or oil core of a microemulsion droplet (10 nm to 500 nm). Complete envelopment of the nucleic acid in a stable microemulsion droplet will protect the fragment from in vivo degradation by nucleases until it can be delivered to the target cells of the body. The complete envelopment also prevents the nucleic acid from being stripped from the droplet and left on the cell surface as the droplet penetrates the cell membrane (by, for example, chemical microporulation). Destabilization of the internalized, microemulsion then releases the entrapped nucleic acid within the cell cytosol, nucleus or other intracellular organelle, which the microemulsion droplets may have penetrated in random distribution throughout the cell. The nucleic acid or other bioactive agent, which is delivered to this intracellular site, is then free to exert it's biological effect.

The variation of nucleic acid size with different constructs for therapy of different diseases will require different size and formulations of the droplets, to obtain optimal cell transfection and therapeutic efficacy. It is necessary to evaluate the ability of the condensation technology used for a specific construct to reduce it to a size that will be enclosed within the lipid core of the microemulsion droplet. Electron microscopy has proved to be a rapid and reliable method to obtain qualitative and quantitative data on the size and structure of the constructs designed to be enclosed within the droplets. The most successful method for achieving this evaluation has been described by Kleinschmidt, A. K. and Zahn, R. K. (1968) In Methods in Enzymology, Grossman, L. and Moldave, K. (eds), Academic Press Inc., New York and London, Vol. 12B, p. 361.

A modification of the Kleinschmidt technique is reported by Coggins, L. W. (1987) in Electron Microscopy in Molecular Biology: A Practical Approach, Sommerville, J. and Scheer, U. (eds), IRL Press, Oxford and Washington D.C., Ch. 1, p. 1-29. In this method, a solution of the nucleic acid construct and a basic protein is used to form a monolayer at a water-air interface. The resulting complex is absorbed to a supporting film mounted on an electron microscopy (EM) grid. The calibrated EM images of the construct reveals the supercoding morphology and the size measurements are determined by a computerized morphometric analysis system.

The negatively charged nucleic acid construct to be evaluated is mixed with cytochrome c (basic protein) and spread via a glass slide ramp onto the surface of a dilute salt solution as described in the prior art (See Davis, R. W. et al. (1980), In A Manual for Genetic Engineering: Advanced Bacterial Genetics, Cold Spring Harbor Laboratory press, Cold Spring Harbor, New York, p. 190-191). The nucleic acid molecules are held in a relaxed two-dimensional conformation in a surface-denatured film of protein, with polar groups tending to face the water and non-polar groups the air. The nucleic acid-protein complex is adsorbed to carbon support film on a copper EM grid. The grid is then stained with uranyl acetate and rinsed in 95% ethanol. Low-angle rotary shadowing of the grids with evaporated platinum is done in an evacuated shadowing apparatus. Both film and digital images are collected of the EM images of the construct at 10,000 to 100,000× magnifications.

Size measurements of the constructs are performed using computerized morphometric analysis. The film images are digitized and analyzed using Image-Pro Plus software (Media Cybernetics, Silver Spring, Md.). Definition of areas for analysis is defined by computerized autotracing or by tracing with a SummaSketch III tracing tablet. Morphometric data from the Image-Pro Plus software is automatically downloaded to Microsoft Excel database spreadsheets or equivalents for descriptive statistical evaluation.

Condensation methodologies will allow the efficient packaging of nucleic acid into droplets of the present invention wherein the nucleic acid is near native form, the potential repulsive charges of the nucleic acid are minimized, and the droplet may incorporate nucleic acids in a minimal droplet size. Indeed, nucleic acids are generally contained in cells in a tightly packaged state which typically occupies 10⁻⁴ to 10⁻⁶ of the volume of the uncondensed nucleic acid within the cell. Nucleic acid may be either supercoiled or unwound enzymatically. For example, such enzymes as topoisomerase, DNA gyrase, and ligase may be used to alter the supercoiled state of circular nucleic acid polymers. Other nucleic acid condensation methods suitable for use in the present invention include without limitation the use of multivalent cations, trivalent cations, lipopolyamine, lipospermine/polyethylenimine, polyamines, and synthetic nucleic acid condensing peptides. The nucleic acid is expected to form condensed aggregates when approximately 90% of the nucleic acid charge is neutralized by the counterions. Such condensed nucleic acid particles are expected to exhibit an orderly, toroidal or rod-like shape and size similar to that of nucleic acid gently released from cells and allow efficient incorporation into the composition of the present invention.

To deliver nucleic acids in near native form, as well as to reduce potential repulsive charges, vis-à-vis the cellular membrane, DNA (either as intact genes, anti-sense DNA, or RNA) may be condensed in-vitro at low salt by the naturally occurring polyamines spermidine3+ and the spermidine4+, by hexamine cobalt (III), as well as by Mg²⁺ initially in methanol water mixtures prior to complexing with the compositions of the present invention. Such multivalent cation condensation procedures may be conducted according to established procedures (see, e.g., Bloomfield, V. A., Biopolymers 31: 1471-1481 (1991)).

Another condensation procedure includes the use of agents such as methyl spermidine in which the amine groups of spermidine are exhaustively methylated (plum, G. E., et al., Biopolymers 30: 631-643 (1990)). Those reagents may be used to condense both DNA and RNA constructs prior to their incorporation into microemulsions.

Plasmids containing clinically relevant genomic or cDNA may be complexed with lipopolyamine micelles whose average diameter (5 nm) has previously revealed three domains (i.e., negative, neutral, and positively charged complexes) depending on the lipopolyamine/plasmid DNA ratio (Pitard, B. et al., Proc. Natl. Acad. Sci. USA 94: 14412-14417 (1997)). Neutral complexes characterized by their full condensation sandwiched between lipid bilayers may be used to formulate the various microemulsions. Similarly, plasmids containing clinically relevant genomic or cDNA may be condensed with either lipospermine or polyethylenimine (Dunlap, D. D., et al., Nuc. Acids. Res. 25: 3095-3101 (1997)). It is anticipated that polyethyleneamine will condense DNA to a more compact form than lipospermine, and thus may be more suitable for particular genetic constructs. Additionally, polyamine headgroups have been known to condense nucleic acids and assist in binding to the cell surface (Remy, J. S., et al., Bioconjug. Chem. 5:647-654 (1994)). Hence, polyamines may be used to condense nucleic acids prior to their combination with a delivery vehicle of the present invention.

Other nucleic acid condensing procedures that employ synthetic nucleic acid condensing peptides may be used to both encourage close packing and condensation, as well as to provide enhanced lipopohilicity to the composition of the present invention. These procedures may stabilize genetic constructs within the droplets or aid in partitioning the nucleic acid(s) into the lipid phase of the droplets. Consensus peptides that have demonstrated DNA and the chromatin condensing properties include without limitation the octapeptide present in many histone H1 subtypes and the imperfect16-mer repeat of the histone H1d C-terminus (see, e.g., Khadake, J. R. et al., Biochemistry 36: 1041-1051 (1997)). Nucleic acid constructs known to be A-T rich may be condensed with synthetic condensing peptides, as described above, prior to incorporation into microemulsions.

Another procedure to condense DNA prior to their incorporation into microemulsions includes the use of peptide analogs called peptoids. Peptoids, like peptides, can assume helix conformations in both aqueous and organic solutions. Peptoids can have monomer sequences that closely resemble those in proteins, but there is no backbone hydrogen bond. Simple helices can be formed in peptoids and they are relatively stable despite their lack of intramolecular hydrogen bonds. Higher molecular weight peptoid oligomers have been to condense and deliver plasmid DNA to cells in vitro. Electron microscopy showed that a 36-mer peptoid complexed with DNA formed spherical structures with diameters of 50 to 100 nm. This size of condensed DNA structure would fit within the lipid core of the microemulsions of the present invention enhancing in vitro and in vivo intracellular delivery characteristics of the DNA. See, e.g., Folding and Design, 2, 369 (1997); Proc. Natl. Acad Sci. USA, 95, 4303 (1998); Proc. Natl. Acad Sci. USA, 95, 1517 (1998).

Nucleic acids of interest may be inserted into a wide range of expression vectors (i.e., plasmids) that may be subsequently used in the present compositions and methods. Accordingly, the nucleic acids, or nucleotide polymers, can be polymers of nucleic acids including genomic DNA, cDNA, mRNA or oligonucleotides containing nucleic acid analogues, for example, the antisense derivatives described in a review by Stein et al., Science 261:1004-1011 (1993) and in U.S. Pat. Nos. 5,264,423 and 5,276,019. Multiple genetic sequences may also be used on a single expression vector. Non-encoding sequences may also be present, to the extent that they are necessary to achieve appropriate expression. As used herein, the term “expression” refers to the transcription of DNA, and the splicing, processing, stability, and optionally the translation of the corresponding mRNA transcript. Still further, the nucleic acids may contain transcriptional and translational regulatory sequences including promoter sequences and enhancer sequences. Thus, the DNA that may be delivered in the compositions of the present invention may be expressed continuously or transiently in host cells, depending on the regulatory elements present.

The transcriptional promoters and enhancers that may be used in conjunction with the nucleic acid of interest include without limitation the herpes simplex thymidine kinase promoter, cytomegalovirus promoter/enhancer (CMV^{P+E}), SV40 promoter, retroviral long terminal repeat promoter/enhancer (LTR^{P+E}), and any permutation and variation thereof. These constructs may be generated using well established molecular biology techniques (see, e.g., Sambrook et al., Molecular Cloning Vols. I-III, Cold Spring Harbor Laboratory Press, Cold Spring Harbor, New York (1989), and Current Protocols in Molecular Biology, John Wiley & Sons, all Vols. (1989) and periodic updates thereof). Promotor/enhancer sequences may also be selected to provide tissue-specific expression.

The nucleic acid of interest may be carried a label for the purpose of providing clinical diagnosis relating to the presence or absence of complementary nucleic acids. Suitable labels are known in the art and include, without limitation, radioactive label, fluorescent label and chemiluminescent label. A genetic marker, also known as a reporter, may also be incorporated with the nucleic acid of interest. Suitable genetic markers include without limitation the lacZ, lux, and cat genes.

Typically, the nucleic acids incorporated in the compositions of the present invention may be administered to a subject for the purpose of introducing or repairing point, missense, nonsense, or other mutations. Similarly, the nucleic acids may be administered to inhibit or enhance the expression of a specific gene or group of genes of interest. Alternatively, the nucleic acid of interest may further incorporate a suicide signal that allows for the controlled elimination of cells harboring and expressing the nucleic acid delivered in the compositions of the present invention. For example, the thymidine kinase (tk) gene may be incorporated with the nucleic acid to be delivered, which would allow the practioner to subsequently kill cells expressing the tk gene by administering effective amounts of acyclovir, gangcyclovir, or the conceptual or functional equivalents thereof.

The nucleic acids used in the present method can be isolated from natural sources, obtained from such sources as ATCC or GenBank libraries, or prepared by synthetic methods. Synthetic nucleic acids can be prepared by a variety of solution or solid phase methods. Generally, solid phase synthesis is preferred. Detailed descriptions of the procedures for solid phase synthesis of nucleic acids by phosphite-triester, phosphotriester, and H-phosphonate chemistries are widely available. See, e.g., Itakura, U.S. Pat. No. 4,401,796; Caruthers et al., U.S. Pat. No. 4,458,066 and 4,500,707; Beaucage et al., Tetrahedron Lett. 22: 1859-1862 (1981); Matteucci et al., J. Am. Chem. Soc. 103:3185-3191 (1981); Caruthers et al., Genetic Engineering 4:1-17 (1982); Iones, chapter 2, Atkinson et al., chapter 3, and Sproat et al., chapter 4, in Oligonucleotide Synthesis: A Practical Approach, Gait (ed.), IRL Press, Washington D.C. (1984); Froehler et al., Tetrahedron Lett. 27:469-472 (1986); Froehler et al., Nucleic Acids Res. 14:5399-5407 (1986); Sinha et al., Tetrahedron Lett. 24:5843-5846 (1983); and Sinha et al., Nucl. Acids Res. 12:4539-4557 (1984).

Oligonucleotides for use as a bioactive agent or as a gene probe, e.g., in in vitro amplification or hybridization methods, are typically synthesized chemically according to the solid phase phosphoramidite triester method described by Beaucage and Caruthers (1981), Tetrahedron Letts. 22(20): 1859-1862, e.g., using an automated synthesizer, as described in Needham-VanDevanter et al. (1984), Nucleic Acids Res. 12:6159-6168. Purification of oligonucleotides, where necessary, is typically performed by either native acrylamide gel electrophoreses or by anion-exchange HPLC as described in Pearson and Regnier (1983), J. Chrom. 255: 137-149. The sequence of the synthetic oligonucleotides can be verified using the chemical degradation method of Maxam and Gilbert (1980) in Grossman and Moldave (eds.) Academic Press, New York, Methods in Enzymology 65:499-560 or the dideoxy chain termination method of Sanger (1977) in Proc. Nat'l Acad Sci. U.S.A. 74: 5463-67.

The nucleic acid or hydrophobically-modified nucleic acid may be a vaccine. Nucleic acid vaccines may be used to induce a neutralizing antibody response. For example, a DNA vaccine may encode an antigen that induces a neutralizing antibody response. Another example is cDNA encoding single-chain antibodies specific to a particular cell or tissue type, for example, small cell lung carcinomas. Such genetic constructs may be generated by the fusion, in various orientations, of both the V_{L} and V_{H} chains containing complementary determining regions downstream of either a constitutive or inducible promoter, such as CMV^{P+E}. Nucleic acid vaccines may also be used to induce cytotoxic T lymphocyte (CTL) responses and T-helper cell responses. Additionally, nucleic acid vaccines may potentiate pre-existing neutralizing antibody responses, pre-existing CTL responses and pre-existing T-helper responses.

A suitable nucleic acid that may be incorporated into the composition of the present invention is a hydrophobically-modified nucleic acid. The hydrophobically-modified nucleic acid may, or may not, be condensed nucleic acid. In a preferred embodiment, the hydrophobically-modified nucleic acid contains a hydrophobic moiety covalently bound to the nucleic acid. Exemplary hydrophobic moieties include, without limitation, steroids and digoxigenin. Alternatively, the DNA may be hydrophobically-modified by other mechanisms and forces whereby nucleic acid associates with a hydrophobic moiety. For example, the nucleic acid may associate through ionic interactions with cation-containing hydrophobic molecules, to thereby produce a hydrophobically-modified nucleic acid. Hydrogen bonding might also be used to associate nucleic acid with hydrophobic moieties.

In a preferred embodiment, the nucleic acid molecules that will be incorporated into compositions of the present invention may be made more lipophilic, and thus more stable in the droplet, by incorporating the steroid hapten, digoxigenin (DIG). A DIG-labeled deoxynucleotide triphosphate, for example dUTP, may be enzymatically linked to the 3′-end of nucleic acids by terminal transferase. In practice, if using a double-stranded DNA, the 3′-end of both the Watson and Crick strands will be subject to DIG labeling. DIG labeling of DNA or RNA will result in the incorporation of about 100-200 DIG-dUTP residues per 3′-end. Moreover, the amount of DIG-labeled deoxynucleotide triphosphate incorporated into nucleic acids of interest may be increased or decreased by adding less or more unlabeled deoxynucleotide triphosphate, such as dATP, dCTP, dGTP, dTTP or mixtures thereof, with the DIG-labeled deoxynucleotide triphosphate in the terminal transferase reaction. The hydrophobicity of the resulting modified nucleic acids will be reflected by the amount of DIG molecules covalently attached. It is anticipated that increases in hydrophobicity of the molecule will stabilize, as well as partition, nucleic acid to a lipophilic region of the compositions of the invention. Use of DIG-labeled nucleic acids, additionally allows for its immunological detection in various tissues by use of commercially available anti-DIG antibody.

Other modifications of a nucleic acid structure to render it more hydrophobic are known in the art and may be used to provide a hydrophobically-modified nucleic acid according to the present invention. Such modifications include, without limitation, the alteration of the negatively charged phosphate backbone bases (for example, the use of non-charged linking group, rather than phosphate groups, to link together the nucleosides), or modification of the sequences at the 5′ or 3′ terminus with agents such as intercalators and crosslinking molecules. Specific examples of such modifications include oligonucleotide analogs that contain methylphosphonate (see, e.g., Miller, P. S. Biotechnology 2:358-362 (1991), phosphorotionate (see, e.g., Stein, Science, 261:1004-1011 (1993), and phosphorodithioate (see, e.g., Brill, W. K. D. J. Am. Chem. Soc., 111:2322 (1989). Other modifications include incorporation of a polyamide backbone in peptide nucleic acids (see, e.g., Nielson et al. Science 254:1497 (1991)), formacetal (see, e.g., Matteucci, M. Tetrahedron Lett. 31:2385-2388 (1990), carbamate and morpholino linkages as well as others known to those skilled in the art.

An alternative means of rendering nucleic acids more hydrophobic are by attachment of saturated fatty acid(s) such as n-dodecanoic (lauric), n-tetradecanoic (myristic), n-hexadecanoic (palmitic), n-octadecanoic (stearic), n-eicosanoic (arachidic) or n-tetracosanoic (lignoceric), through dUTP (as above), to the 3′-ends of the nucleic acid molecule via terminal transferase. The same saturated fatty acids could be chemically coupled to peptides, and similarly portioned to the lipophilic region of emulsions for therapeutic application of various small peptide molecules.

Another approach to hydrophobically modify nucleic acids is to use molecules that non-covalently associate with nucleic acids by, for example, hydrogen bonding and intercalation. Various DNA intercalating agents may be used to render DNA constructs more hydrophobic, and thereby encourage its association with the lipophilic phase (droplets) of the delivery vehicles of the present invention. As an example, naphthalene imides carrying alkyl chains of different lengths may be synthesized and then used to assess their impact on imparting a hydrophobic effect on DNA by removing the structure-stabilizing water from the DNA groove. See, e.g., Takenaka, S. et al. “Hydrophobic Effect of Alkyl Substituents on DNA Intercalation of a Dye” Nucleic Acids Symp. Ser. 37:105-106, 1997. Additionally, anthryl probe, e.g., 9-(anthrylmethyl)trimethylammonium chloride, may be used to enhance the interaction of microemulsion formulated surfactants and thus elevated hydrophobic nature of the encapsulated DNA. See. e.g., Bhattacharya, S. and Mandal S. S. “Interaction of Surfactants with DNA. Role of Hydrophobicity and Surface Charge on Intercalation and DNA Melting” Biochem. Biophys. Acta. 1323:29-44, 1997),

Additional references relevant to the hydrophobic modification of nucleic acids are: Schmitz, G. G. et al., Anal. Biochem. 192:222-231, 1991; Gebeyehu, G. et al., Nucleic Acids Res. 15:4513, 1987; Khandijan, E. W., Bio/Technology 5:165, 1987; and Collins, M. L. and Hunsaker, W. R., Anal. Biochem. 151:211, 1985.

Bioactive agents other than nucleic acids may be desirably rendered hydrophobic prior to incorporation into a composition of the present invention. For example, it may be desirable to covalently bind DIG to the FC portion of an antibody, so that the bonding portion of the antibody protrudes from a droplet having a hydrophobic interior. Other bioactive agents that may be covalently bound to hydrophobic molecules include, without limitation, antibodies, Fab or other single chain antibodies, peptide ligands, or peptide chimeras. The present invention provides for this option.

In general, the delivery vehicle of the invention comprising an oil, an oil-immiscible compound and a surface active agent. The compositions of the invention (excluding the bioactive agent), should be of low or non-toxicity to the cell. Toxicity for any given delivery vehicle may vary with the concentration of components in the vehicle. It is also beneficial if the vehicle components are metabolized or eliminated by the host, and that such metabolism or elimination may be done in a manner that will not be harmfully toxic.

In another aspect, the invention provides a composition which includes a nucleic acid, an oil, an oil-immiscible compound, a noncationic surface active agent, and essentially no cationic lipid. In one embodiment, the composition contains no cationic lipid.

In another aspect, the invention provides a composition which includes a hydrophobically-modified nucleic acid, an oil, an oil-immiscible compound and at least one surface active agent. In one embodiment of the invention, the surface active agent is non-cationic.

In another aspect, the invention provides a method of forming a composition which includes a bioactive agent, where the method includes the steps of (a) combining the bioactive agent, an oil component, an oil-immiscible component, and at least two noncationic surfactants; and (b) mixing the combination of step (a) to form the composition.

In another aspect, the invention provides a method of forming a composition which includes a bioactive agent, where the method includes the steps of (a) combining the bioactive agent, an oil component, and oil-immiscible component, and a non-cationic surface active agent in the substantial absence of a cationic lipid, and (b) mixing the combination of step (a) to form the delivery composition.

In still other embodiments, the compositions will have a targeting moiety attached to the surface of the droplet. In one aspect, a targeting molecule may be present, where suitable targeting molecules include antibodies, Fab or other single chain antibodies, peptide ligands, or peptide chimeras. Methods of attaching targeting moieties (e.g., antibodies, proteins) to lipids (such as those which may be used in the present droplets) are known to those of skill in the art.

In the above compositions, the surface active agent may encompass a first and second surface active agents of non-identical structures. In these systems, the first surface active agent may have an HLB of 8 or less and the second surface active agent may have an HLB of 10 or greater. The first and second surface active agents may both be nonionic, preferably having no charge groups. In a further embodiment, the surface active agent may encompass a first, second and third surface active agents of non-identical structures.

In a preferred embodiment of the above compositions, the delivery vehicle does not contain an effective amount of a cationic surfactant, and more preferably contain no cationic surfactant. Thus, a preferred embodiment of the invention provides a composition containing a nonionic surfactant primarily residing in an oil phase, and a nonionic surfactant primarily residing in the oil-immiscible phase, where the nonionic surfactant preferably contains no charges, and certainly contains no net charge. As used herein, whether a surface active agent has a charge is determined at physiological pH, that is, a pH of about 6.5 to 7.5.

The systems according to the invention can also contain stabilizers. Apart from antioxidants and preservatives, these can also be buffer substances and isotonic agents. Furthermore, for the chemical stabilization of the bioactive agent or bioactive agent mixture, it is possible to incorporate special stabilizers, such as, e.g., tartaric acid, in conjunction with ergotamine tartrate or sodium polyphosphate, in conjunction with phenyl butazone.

Additionally, the composition may include oil-protective agents which protect oils against free-radical and oil-peroxidative damages on storage. Lipophilic free-radical quenchers, such as alpha-tocopherol and water-soluble iron-specific chelators, such as ferrioxamine, are suitable.

The present invention also provides therapeutic compositions in kit form. The kit will typically be comprised of a container which is compartmentalized for holding the various elements of the kit. The kit will contain the compositions of the present inventions, preferably in dehydrated form, with instructions for their rehydration and administration.

Any of the above compositions may be in the form of an emulsion, in the form of a microemulsion, or in the form of a liquid crystal. Any of the above compositions may be self-emulsifying. A preferred composition contains droplets, which may also be referred to as particles or microspheres. In a typical composition, greater than about 90% of the droplets have a diameter within the range of about 0.1 nm to about 1 millimeter, preferably about 0.1 nm to about 1 micrometer, more preferably about 0.1 nm to about 500 nm. In a typical composition, greater than about 90% of the droplets have a diameter within the range of about 50 nm to about 250 nm, while in another typical composition, greater than about 90% of the droplets have a diameter within the range of about 100 nm to about 150 nm.

The invention thus provides compositions having an oil continuous phase, which may taken the form of a water-in-oil (W/O) emulsion or microemulsion. Additionally, the invention provides compositions having an aqueous continuous phase, which may take the form of an oil-in-water (O/W) emulsion or microemulsion. Additionally, the invention provides compositions which are bicontinuous. Thus, the present invention provides composition that contain at least two distinct phases. As used herein, “droplets” will refer to the bioactive agent-enriched phase, even though, as in the case of a bicontinuous composition, spherical or even semi-spherical droplets may not be visible or present in the composition. Preferably, the composition is biocompatible.

Regular emulsions are also called conventional emulsions, macroemulsions, and coarse emulsions. Regular emulsions are mixtures of an oil(s), an oil-immiscible compound, and a surface active agent to stabilize the dispersed phase. The size of the disperse phase of regular emulsions typically range from about 200 nm to 1 mm. As a result, regular emulsions are typically white and opaque. Regular emulsions usually require significant energy input to make them. This energy input can be generated by homogenizers and high shear stirrers or by sending pressurized liquids through small orifices (i.e., microfluidizers). Regular emulsions are not thermodynamically stable, although if given the proper components and conditions these emulsions may be stable for several years. Note that regular emulsions that have been “micronized” by the input of significant amounts of energy are not equivalent to microemulsions except possibly in droplet size. The properties of microemulsions are quite different from the properties of regular emulsions.

The microemulsion delivery vehicles can be o/w, w/o or bicontinuous and are characterized as being self-emulsifying, dispersions of oil and an oil-immiscible material stabilized by interfacial films of surface-active agents. That is, the microemulsions form spontaneously without the need for energy input. The microemulsions are further generally characterized by small average droplet sizes, from about 0.1 nm to about 200 nm, and in one embodiment have a diameter of less than about 100 nm. They arc further characterized by their wide range of temperature stability, typically from about 5° C. to about 100° C., and they appear to be thermodynamically stable. Microemulsions are also relatively insensitive to pH or ionic strength of an aqueous phase when nonionic surfactants are used. Furthermore, the microemulsions are usually transparent or opalescent when viewed by both macroscopic and microscopic means. Undisturbed, they are optically isotropic when examined under polarized light. The microemulsions typically require a second interfacial film-stabilizing agent or co-surfactant. The co-surfactant can be a second surfactant of differing HLB than the primary surfactant, short-chain alcohol or ester, carboxylic acid, organic amine, etc. Bicontinuous structures exist where there are similar amounts of oil and oil-immiscible compound present and may exist as intermediate structures between w/o and o/w microemulsions.

For a further discussion of microemulsions, see, e.g., M. Kahlweit, Science, 240:617-621 (1988); Microemulsions: Theory and Practice, Prince (ed.). Academic Press, New York (1977); and Industrial Applications of Microemulsions, Solans and Kunieda (eds.), Marcel Dekker, Inc., New York (1997). For a general discussion of microemulsions, see, e.g., Danielsson, I. et al., Colloids and Surfaces 3:391-392 (1981); Bhargava, H. N. et al. Pharmaceutical Technology, 46-54 (March 1987); Friberg, S. et al., Progr. Colloid & Polymer Sci. 56:16-20 (1975); Kahlweit, M. et al., J. Colloid and Interface Sci. 118(2):436-453 (August 1987); Benita, S. et al., J. Pharmaceutical Sciences 82(11):1069-1079 (1993); Ruckenstein et al., J. Colloid and Interface Science 76(1):188-200 (July 1980); Ruckenistein et al., J. Colloid and Interface Science 75(2):476-492 (June 1980); Bansal, V. K. et al., J. Colloid and Interface Science 75(2):462-475 (June 1980); and Mukherjee S., J. Colloid and Interface Science 91(1):223-243 (January 1981). A delivery vehicle in the form of a microemulsion is a preferred embodiment of the present invention. The delivery vehicle contains droplets having an average diameter of 0.1-200, preferably 0.1-100 nm.

The compositions of the invention may also take the form of other phase structures, or mixtures of structures, such as various liquid crystal (e.g., lamellar, hexagonal, or cubic), gel and solid phases that, when administered in vitro or in vivo, may readily convert to the above mentioned water-in-oil emulsion, oil-in-water emulsion, or bicontinuous phase due to dilution by cell media or other biological fluid. A microemulsion embodiment of the present invention may be converted to a regular emulsion embodiment upon dilution prior to administration, or upon administration into an aqueous environment either in vitro or in vivo. For instance, a liquid crystal gel may convert, upon exposure to an aqueous environment, to microemulsion droplets.

In a preferred embodiment of this invention, the composition will be a microemulsion. A microemulsion composition of this invention may be preferred for several reasons. For example, a microemulsion is self-emulsifying, thus eliminating the need for significant energy input which may damage or degrade the bioactive agent, especially higher molecular weight nucleic acids. A microemulsion is thermodynamically stable. A microemulsion composition of this invention may have improved shelf-life over a regular emulsion composition. Because microemulsions are usually transparent, one can readily determine whether the bioactive agent has aggregated or precipitates have formed in the product. In fact, the appearance of clarity for a microemulsion indicates that the microemulsion is in a thermodynamically-stable form. Preferred delivery vehicles, with or without a bioactive agent, are transparent, rather than cloudy. Upon dilution, the transparent microemulsions of the present invention will typically become cloudy. In the “transparent” form, the microemulsions of the present invention have excellent storage stability. The typically small droplet size of a microemulsion allows for interstitial penetration and for dissemination of droplets throughout tissues and organs. Also, the typically small droplet size of a microemulsion allows for droplets to be more easily taken into a cell structure.

In some instances, it may be desirable to employ a regular emulsion in a method of the present invention. Microemulsions typically require relatively greater amounts of the surface active agent to stabilize the disperse phase than do regular emulsions. If low levels of the surface active agent(s) are required, a regular emulsion composition of the invention would be desirable. If long stability or degradation of the bioactive agent are not a concern, then a regular emulsion might be desirable as well.

The art of constructing a regular emulsion is well known and will not be described here. By way of reference, further discussion on the construction of emulsions can be found in Emulsion Science, Sherman (ed.), Academic Press, New York (1968).

The specific bioactive agent to be incorporated into the composition will also impact on the form which the composition takes. The solubility properties of the bioactive agent will determine whether a water-in-oil or oil-in-water emulsion or microemulsion should be used. The size and molecular weight of the agent may also have a bearing on the composition.

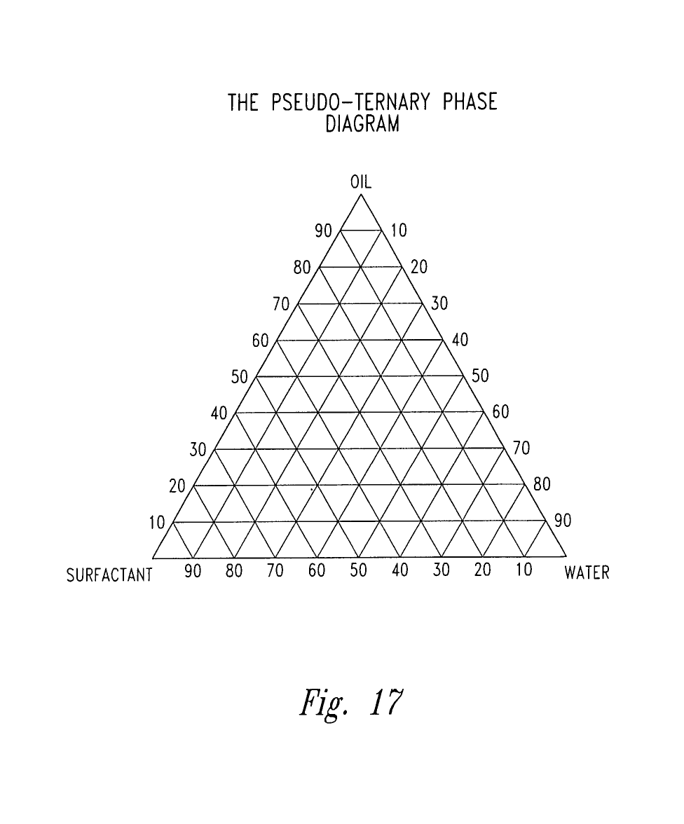

The use of the pseudo-ternary phase diagram (see FIG. 17) is a helpful tool in the development of compositions of this invention. Principles behind the preparation of such pseudo-ternary phase diagrams are well known in the art. The pseudo-ternary phase diagram can be used to locate compositions of components that result in microemulsions, regular emulsions, and other phase structures (e.g. liquid crystal phases). Once the various components have been chosen, a pseudo-ternary phase diagram can be constructed which will describe all the phases resulting from combinations of the components of a given composition of the invention. The construction of a pseudo-ternary phase diagram is more fully described in the above references and in Microemulsion Formulation Guide, Gattefossé S. A. (1992).

The use of various non-ionic surfactants may be used to influence the size of an emulsion droplet. Thus, the encapsulation of DNA may be optimized through use of various non-ionic surfactants. We have observed that upon mixing of DNA with emulsions, the droplet size increases to differing levels depending upon the type of non-ionic surfactant used in the emulsion preparation. Droplet size may need to be selected in view of the size of the bioactive agent. Droplet size may be adjusted by proper selection of the non-ionic surfactant used in the composition. As an example, it has been previously demonstrated that a 5- to 14- fold increase in diameter of the lipid droplets (DNA/emulsion complexes) was observed when DNA was mixed with emulsions containing Span, Brij 72™, Brij 74™, Brij 76™, Pluronic F68™ or Pluronic F127™. In regard to droplet size, see, e.g., Liu, F., et al., Pharmaceutical Res. 13:1642-1646, 1996.

When droplets are present in the composition of the invention, the concentration of droplets can vary widely, i.e., from less than about 0.05%, usually at or at least about 2 to about 5% to as much as about 10 to about 60% by weight and will be selected primarily by fluid volumes, viscosities, etc., in accordance with the particular mode of administration selected. For example, the concentration may be increased to lower the fluid load associated with treatment. This may be particularly desirable in patients having atherosclerosis-associated congestive heart failure or severe hypertension. Alternatively, droplets composed of irritating lipids may be diluted to low concentrations to lessen inflammation at the site of administration. Typically, the concentration of the PEG, PEG-ceramide or G_{M1}-modified lipids in the droplet will be about 1-15%.

The individual components of the pharmaceutical compositions may be sterilized by conventional, well-known sterilization techniques. Aqueous solutions can be packaged for use or filtered under aseptic conditions and lyophilized, the lyophilized preparation being combined with a sterile aqueous solution prior to administration to reconstitute a composition of the invention, e.g., a microemulsion. The compositions can contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, and calcium chloride.

The compositions of the present invention are useful for the introduction of nucleic acids and other bioactive agents into cells. Accordingly, the present invention also provides methods for introducing a nucleic acid (e.g., a plasmid) into a cell. The methods are carried out, for example, in vitro, in vivo, ex vivo, or in situ by first forming the compositions as described above, then contacting the composition with the cells for a period of time sufficient for transfection to occur.

The droplet-containing compositions of the present invention (which may also be referred to as particle-containing) can be adsorbed to almost any cell type with which they are mixed or contacted. Preferably, the droplets deliver their bioactive agent into the cell through chemical microporulation. Alternatively, after the droplets have been adsorbed onto the cell, the droplets can enter the cells by being endocytosed by a portion of the cells, exchange lipids with cell membranes, or fuse with the cell membrane. When fusion takes place, the droplet membrane is integrated into the cell membrane and the contents of the droplet combine with the intracellular fluid. Transfer or incorporation of the nucleic acid portion of the droplet can take place via any one of these pathways.

As used herein, “microporulation” refers to process wherein an interaction occurs between surface active agent(s) of an emulsion or microemulsion composition and the membrane of a cell or an intracellular organelle, such that a transient pore is formed in the membrane that allows one or more droplets of the emulsion or microemulsion to pass into the interior of the cell or organelle. The terms “microporulation”, “chemical microporulation” and “microporation” may herein be used interchangeably. Electron micrographs demonstrate that compositions of the invention may contact and directly penetrate a cellular membrane by separating the phospholipid bilayer and forming a temporary pore that allows an intact droplet to pass into the cell cytosol. The pore is small, typically the width of a microemulsion droplet (about 10-500 nm in diameter), and seals almost immediately after the droplet passes through the membrane. The rapid sealing of the pores and the selection of composition components having low cytotoxicity allows the claimed compositions to deliver bioactive agents into cells with no or tolerable deleterious effects on a cell's physiological function or viability. The stable emulsion or microemulsion droplets remain intact and upon contact with intracellular membranes, penetrate through them by microporulation, allowing the droplets to enter the nucleus and other intracellular organelles such as mitochondria, the smooth and rough endoplasmic reticulum, Golgi apparatus vesicles, etc., where they can then release a bioactive agent. The release of the bioactive agent, with subsequent action on the cell, may be slow due to the fact that the emulsion or microemulsion droplets are fairly robust. Thus, the microporulation process of the present invention may provide for the controlled release of the bioactive agent, which may be desirable when, for example, the bioactive agent is beneficial during a particular stage in the cell cycle, for example, during division.

In contrast, other non-viral vectors such as polymer droplets, cationic lipid emulsions and polymer filaments attach to the cell surface and enter the cell by fusion, or by endocytosis or phagocytosis where the carrier is present in an endocytic vesicle. In many instances, the bioactive agent needs to be released from the carrier and escape from the vesicle to escape degradation by vesicular enzymes, and enter the cell cytosol to become effective. Electron micrographs demonstrate that the cationic non-viral gene delivery agent lipoplex (Philip L., Felgner, Nonviral Strategies for Gene Therapy, Scientific American, pp. 102-106, June 1997) enter the cells by this endocytic and phagocytic vesicle uptake method. Electron microscopy studies in our laboratories have demonstrated that the commercial cationic lipid DNA transfection product LipofectAMINE™ (Gibco BRL, Life Technologies, Gaithersburg, Md.) also enters the cells by the same vesicle uptake method. Indeed, studies from other laboratories have reached the same conclusion (see, e.g., Zabner, J. et al. J. Biol. Chem. 270(32):18997-19007, Aug. 11, 1995). Liposomes, another non-viral gene vehicle, appear to fuse with the cellular membrane and release their gene or therapeutic agent directly into the cytoplasm; however, the liposome does not remain intact and deliver the agents directly to the nucleus or cellular organelles. In contrast, the claimed compositions of the present invention deliver the genetic material or other bioactive agent directly into essentially any subcellular compartment, including without limitation the cytosol, nucleus and other cellular organelles.

For in vitro applications, the delivery of nucleic acids can be to any cell grown in culture (primary or immortalized), whether of plant or animal origin, vertebrate or invertebrate, and of any tissue or type. In preferred embodiments, the cells will be animal cells, more preferably mammalian cells, and most preferably human cells.

Contact between the cells and the inventive composition, when carried out in vitro, takes place in a biologically compatible medium. Treatment of the cells with the composition of the invention is generally carried out at physiological temperatures (about 37° C.) for about 1 to 48 hours, preferably for about 2 to 4 hours.

In one group of preferred embodiments, a droplet inventive composition is added to monolayers that are 60-80% confluent and have a cell density of about 10³ to about 10⁵ cells/mL, more preferably about 2×10⁴ cells/mL. The concentration of the droplets added to the cells is preferably about 0.01 to 0.2 μg/mL, more preferably about 0.1 μg/mL.

Alternatively, the compositions of the present invention can also be used for in vivo gene transfer, using methods which are known to those of skill in the art. In particular, Zhu et al., Science 261:209-211 (1993), describes the intravenous delivery of cytomegalovirus (CMV)-chloramphenicol acetyltransferase (CAT) expression plasmid using DOTMA-DOPE complexes. Hyde et al., Nature 362:250-256 (1993), describes the delivery of the cystic fibrosis transmembrane conductance regulator (CFTR) gene to epithelia of the airway and to alveoli in the lung of mice, using liposomes. Brigham et al., Am. J. Med. Sci. 298:278-281 (1989), describes the in vivo transfection of lungs of mice with a functioning prokaryotic gene encoding the intracellular enzyme chloramphenicol acetyltransferase (CAT).

For in vivo administration, the pharmaceutical compositions are preferably administered parenterally, i.e., intraarterially, intranasally, intravenously, intraperitoneally, subcutaneously, or intramuscularly. More preferably, the pharmaceutical compositions are administered intravenously or intraperitoneally by a bolus injection. See, e.g., Stadler et al., U.S. Pat. No. 5,286,634. Intracellular nucleic acid delivery has also been discussed in Straubringer et al., Methods In Enzymology, Academic Press, New York, 101:512-527 (1983); Mannino et al., Biotechiques 6:682-690 (1988); Nicolau et al., Crit. Rev. Ther. Drug Carrier Syst. 6:239-271 (1989), and Behr, Acc. Chem. Res. 26:274-278 (1993). Still other methods of administering lipid-based therapeutics are described in, for example, Rahman et al., U.S. Pat. No. 3,993,754; Sears, U.S. Pat. No. 4,145,410; Papahadjopoulos et al., U.S. Pat. No. 4,235,871; Schneider, U.S. Pat. No.4,224,179; Lenk et al., U.S. Pat. No. 4,522,803; and Fountain et al., U.S. Pat. No. 4,588,578.

In certain embodiments, the pharmaceutical compositions of the invention may be contacted with the target tissue by direct application of the composition to the tissue. The application may be made by topical, “open” or “closed” procedures. By “topical,” it is meant the direct application of the pharmaceutical preparation to a tissue exposed to the environment, such as the skin, oropharynx, external auditory canal, and the like. “Open” procedures are those which include incising the skin of a patient and directly visualizing the underlying tissue to which the pharmaceutical composition is applied. This is generally accomplished by a surgical procedure, such as a thoracotomy to access the lungs, abdominal laparotomy to access abdominal viscera, or other direct surgical approach to the target tissue. “Closed” procedures are invasive procedures in which the internal target tissues are not directly visualized, but accessed via inserting instruments through small wounds in the skin example, the inventive composition may be administered to the peritoneum by needle lavage. Likewise, the pharmaceutical composition of the invention may be administered to the meninges or spinal cord, or cerebral spinal fluid, by infusion during a lumbar puncture followed by appropriate positioning of the patient as commonly practiced for spinal anesthesia or metrazamide imaging of the spinal cord. The inventive compositions may be administered through endoscopic devices, or via an aerosol inhaled into the lungs (see, e.g., Brigham et al., Am. J. Sci. 298(4):278-281 (1989)), or by direct injection at the site of disease (see, e.g., Culver, Human Gene Therapy, MaryAnn Liebert, Inc., Publishers, New York, pp. 70-71 (1994)). The inventive compositions may be administered via an intravascular infusion catheter, e.g, during or following the PTCA procedure described elsewhere herein. This may be useful in treating, for example, restenosis. In one aspect, the present invention provides a method of treating restenosis, optionally by administration of a therapeutic microemulsion composition described herein by intravascular infusion catheter.

In developing a composition of the present invention, the possible route(s) of administration for the composition should first be considered. In a preferred embodiment, the composition will be administered parenterally. In this instance, only components that are pharmaceutically acceptable for parenteral applications should be used. Additionally, the compositions of the present invention may appear in any of a broad range of topical dosage forms including without limitation topical creams, pastes, ointments, gels, lotions and the like. If the composition is to be delivered to the skin, e.g., topically, the number of pharmaceutically acceptable components increases greatly because more chemical compounds are pharmaceutically acceptable for topical application than parenteral application. Other possible routes of administration of the compositions of this invention have been described herein. Thus, whether to include a specific component in a composition of the present invention will depend, at least in part, on whether that component is compatible with the desired route of administration. Some components may be acceptable for more than one route.

Suitable routes of administration include, without limitation, buccal, cerebral (e.g., injection into the ventricle of the brain or submingeal cerebral spinal fluid), gastric, oral, inhalation, intraarterial, intramuscular, intranasal, intraoccular, intraspinal (e.g., into the spinal cord fluid), intravenous, mucosal, parenteral, rectal, sublingual, topical, urethral, urinary bladder and vaginal. The term parenteral as used herein includes subcutaneous injections, intravenous, intramuscular, intradermal, intrasternal injection or infusion techniques. The compositions may be added directly to components of the central nervous system, e.g., the compositions may be injected directly into the brain and spinal parenchyma.

The methods of the present invention may be practiced in a variety of subjects. Preferred subjects include mammalian species, such as humans, non-human primates, dogs, cats, cattle, horses, fish, reptiles, birds, chickens, sheep, turkeys, mice, rats and the like. The subject may be a plant.

The amount of droplets administered will depend upon the amount of nucleic acid in the droplets; the particular nucleic acid used, the disease state being diagnosed; the age, the weight, the condition of the patient, the route of administration and the judgment of the clinician; but will generally be between about 0.01 and about 500 mg per kilogram of body weight; preferably between about 0.1 and about 5 mg/kg of body weight or about 10⁸-10¹⁰ droplets per injection.

In one aspect, the invention provides a composition for transporting a bioactive agent across a biological barrier. The composition includes a bioactive agent, an oil, an oil-immiscible compound and a noncationic surface active agent, and is able to transport the agent across the barrier. Preferably, the bioactive agent is transported across the barrier by chemical microporulation. The biological barrier may be any of a cell wall of a plant cell, a cellular membrane of an animal cell, a nuclear membrane, an organelle membrane such as endoplasmic reticulum, and mitochondrial membrane, a blood-brain barrier, a blood-ocular barrier, a biological barrier present within a living plant, or a biological barrier present within a living animal, where suitable animals include, without limitation, bird, fish, human, livestock, and poultry. In one embodiment, the bioactive agent is a drug. In another embodiment, the bioactive agent is a nucleic acid.

In another embodiment, the invention provides a composition that includes a nucleic acid (as a bioactive agent), and, as the delivery vehicle, a composition that includes an oil, an oil-immiscible compound, a noncationic surface active agent, and essentially no cationic lipid. In another embodiment, the invention provides a composition that includes a nucleic acid (as a bioactive agent), and, as the delivery vehicle, a composition including an oil, an oil-immiscible compound, and two noncationic surface active agents. In one embodiment, the delivery vehicle contains only the recited ingredients.

In the above compositions, the nucleic acid may be any of adenine, guanine, cytosine, thymine, and uracil, and polymers containing a plurality of residues thereof. In one embodiment, the nucleic acid is a gene, while in another embodiment, the bioactive agent is a hydrophobically-modified nucleic acid.

In another embodiment, the invention provides a composition that includes a hydrophobically-modified nucleic acid (as a bioactive agent), and, as the delivery vehicle, a composition including an oil, an oil-immiscible compound and at least one surface active agent. In a preferred embodiment, the surface active agent is noncationic, i.e., does not carry a net positive charge, and preferably does not contain any positive charge.

In any of the above compositions, the hydrophobically-modified nucleic acid contains a hydrophobic moiety associated with the nucleic acid. The hydrophobic moiety may be associated by any one or more of covalent bonding, ionic bonding, or hydrogen bonding. In one embodiment, the hydrophobic moiety is selected from steroids and digoxigenin.

In the above-compositions, the bioactive agent may be a nucleic acid vaccine. The hydrophobically-modified nucleic acid may likewise be a hydrophobically-modified nucleic acid vaccine. A suitable nucleic acid vaccine is a DNA vaccine. Another suitable nucleic acid vaccine is capable of inducing at least one of a cytotoxic T-lymphocyte (CTL) response, a T-helper response, and a neutralizing antibody response in a subject to whom the composition is administered. Another suitable nucleic acid vaccine is capable of potentiating at least one of a pre-existing CTL response to an antigen, a preexisting T-helper response to an antigen, and a pre-existing neutralizing antibody response to an antigen, in a subject to whom the composition is administered. As before, any of the nucleic acid vaccines may be hydrophobically modified.

In any of the above composition, there may be present, in addition to the other indicated components, a targeting molecule. As used here and throughout this description, “a” means “at least one”, and is not to be construed as referring to only one. In various embodiments of the invention, the oil of the composition may be a vegetable oil. Suitable vegetable oils may be selected from castor, coconut, corn, ethereal, olive, palm, peanut, rape, and sesame oils. The oil is preferably miscible with water to an extent of less than about 0.1 g oil per about 100 g water at 25° C., and is more preferably essentially immiscible with water or other aqueous composition.

In the above compositions, the oil-immiscible compound preferably includes water, so as to form an aqueous solution. Preferably, the oil-immiscible compound is soluble in corn oil to an extent of less than about 0.1 g oil-immiscible compound per about 100 g corn oil at 25° C.

In various embodiments of the invention, the noncationic surface active agent of the compositions described above is a nonionic surface active agent. As used herein, nonionic surfactant does not have any net charge at physiological pH (i.e., pH of about 6.5-7.5), and preferably does not have any charge at all. In another embodiment, the noncationic surface active agent is an anionic surface active agent. An anionic surfactant contains at least one negative charge, and preferably has a net negative charge. In the compositions, polyethylene glycol is a favored component of the noncationic surface active agent. Another favored component for a noncationic surface active agent is polyglycerol.

In various embodiments, the compositions described above may contain first and second surface active agents of non-identical structures. Preferably, the structurally non-identical surface active agents belong to different classes of a type (cationic, anionic or nonionic) surface active agent. In certain embodiments, the first surface active agent has a hydrophilic-lipophilic balance number (HLB) of 8 or less and the second surface active agent has a HLB of 10 or greater. In other certain embodiments, the first and second surface active agents are both nonionic.

In preferred embodiments for the afore-described compositions, the delivery vehicles do not contain, or are not prepared from, any cationic surface active agent. The bioactive agent, however, when it is condensed and/or hydrophobically-modified, may include a cationic surfactant which neutralizes anionic charge(s) on the bioactive agent. In other preferred embodiment, the delivery vehicle includes a nonionic surfactant which is primarily dissolved in an oil phase, and a nonionic surfactant which is primarily dissolved in the oil-immiscible phase.

The compositions of the invention may be in the form of an emulsion. In another embodiment, the compositions are in the form of a microemulsion. In one embodiment, the microemulsion contains droplets, wherein greater than about 90% of the droplets have a diameter within the range of about 0.1 nm to about 500 nm. In a preferred embodiment, greater than about 90% of the droplets have a diameter within the range of about 10 to 200 nm.

In certain embodiments of the invention, the delivery vehicle is self-emulsifying. The composition may have an oil continuous phase. The composition may be an water-in-oil (W/O) emulsion or microemulsion. In other embodiments, the compositions has an aqueous continuous phase. The composition may be an oil-in-water (O/W) emulsion or microemulsion. In certain other embodiments, the composition is bicontinuous. Regardless of its form, the composition is preferably biocompatible.

The compositions described above will typically deliver the bioactive agent by microporulation across both a cellular membrane and a nuclear membrane.

In another aspect, the invention provides a method of transporting a bioactive agent across a biological barrier, where the method includes the steps of (a) contacting a composition which includes the bioactive agent with a biological barrier; and (b) transferring the bioactive agent across the barrier by chemical microporulation. In the above methods, the bioactive agent may be a nucleic acid or a hydrophobically-modified nucleic acid.

In another aspect, the invention provides a method of treating a plant or animal subject in need thereof, where the method includes the step of administering at least one of composition (a), (b) and (c) to the subject, where (a) is a composition that includes a nucleic acid, an oil, an oil-immiscible compound, a noncationic surface active agent, and essentially no cationic lipid; (b) is a composition that includes a nucleic acid, an oil, an oil-immiscible compound, and at least two noncationic surface active agents; and (c) is a composition that includes a hydrophobically-modified nucleic acid, an oil, an oil-immiscible compound and at least one surface active agent.

In the above methods, the nucleic acid or hydrophobically-modified nucleic acid preferably enters a cell of the subject by chemical microporulation. Preferably, the microporulation delivers agent across both a cellular membrane and a nuclear membrane. The cells may be contacted in vitro with the composition that includes the bioactive agent or nucleic acid, the contacting being for a period of time sufficient to achieve delivery of the bioactive agent into the cell, and then the contacted cells are introduced to the subject. When the composition is used to deliver the bioactive agent to a viable cell by microporulation, the microporulated cell preferably remains viable for at least 48 hours after the microporulation. The cells may be of animal or plant original, and the animal may be a vertebrate or invertebrate, where suitable vertebrates include, without limitation, humans, non-humans, cats, dogs, sheep, cattle, fowl, pig, rat, mouse and horses.

In the inventive methods, the contacting typically takes place within a biologically compatible medium. A typical contacting takes place for a time of about 5 minutes to 48 hours and a temperature of about 25 to 45° C. Often a contact time of about 15 minutes is sufficient to achieve delivery of the active agent into the cell.

The cells may be contacted in vivo with the composition that includes the bioactive agent or nucleic acid, where the contacting is for a sufficient period of time for the bioactive agent to enter the cells of the subject, and preferably for transfection to occur. In this case, the contacting may be accomplished through parenteral administration of the composition to the subject, where the parenteral administration may be, for example, intravenous or intraperitoneal injection. The contacting may be accomplished through delivering the bioactive agent or nucleic acid to the epithelia of the subject's airway or to alveoli in the subject's lung. The contacting may be accomplished through topical administration of the composition to the subject. The contacting may be accomplished by placing the composition within an opening formed through incision of the subject's skin.

In the methods, the bioactive agent may be a nucleic acid or a hydrophobically-modified nucleic acid. The bioactive agent may be a nucleic acid vaccine, such as a DNA vaccine. In the methods, the bioactive agent typically, and in a preferred embodiment, enters a cell of the subject by chemical microporulation. In the methods, the cells may be contacted in vitro with the composition comprising the bioactive agent, the contacting being for a sufficient period of time for transfection to occur, and the thus contacted cells are introduced to the subject. The cells may be of animal origin. The contacting should typically take place for a time of about 5 minutes to about 48 hours and a temperature of about 25° C. to about 45° C.

The cells may be contacted in vivo with the composition including the bioactive agent, where the contacting is for a sufficient period of time for delivery of the bioactive agent into one or more cells. In one embodiment, the contacting may be accomplished through parenteral administration of the composition to the subject In any of the methods, the cell may be a smooth muscle cell. Preferably, the contacting introduces integration of a functional copy of a gene into a chromosome of the subject. For example, in one embodiment, the contacting introduces a DNA fragment of a gene into the nucleus of the cell.

The bioactive agent may be a nucleic acid vaccine that induces an immune response in the subject. The bioactive agent may be a nucleic acid vaccine that induces at least one of a CTL response, a T-helper response, and a neutralizing antibody response. In any of the methods, the bioactive agent may be a nucleic acid vaccine that potentiates at least one of a pre-existing CTL response to an antigen, a pre-existing T-helper response to an antigen, and a pre-existing neutralizing antibody repines to an antigen.

In another embodiment, the present invention provides a method of preparing a composition for transporting a bioactive agent across a biological membrane. The method includes: (a) modifying a nucleic acid to provide the bioactive agent, the bioactive agent being more hydrophobic than the nucleic acid; and (b) combining the bioactive agent with a composition selected from (i) an oil, an oil-immiscible compound, a noncationic surface active agent, and essentially no cationic lipid; and (ii) an oil, an oil-immiscible compound, and two noncationic surface active agent. In another embodiment, the nucleic acid need not be modified. In another embodiment, the nucleic acid is combined with any of the delivery compositions of the present invention.

The compositions and methods of the present invention may be used to treat a wide variety of cell types, in vivo and in vitro. The cells may be somatic or germ line cells. Among those most often targeted for gene therapy are actively dividing cells, such as hematopoietic precursor (stem) cells. Other cells include those of which a proportion of the targeted cells are nondividing or slow dividing. These include, for example, circulating fibrocyte stem cells, endothelial stem cells or other mesenchymal stem cells; fibroblasts, keratinocytes, endothelial cells, skeletal and smooth muscle cells, osteoblasts, neurons, quiescent lymphocytes, terminally differentiated cells, slow or non-cycling primary cells, parenchymal cells, lymphoid cells, epithelial cells, bone cells, a cell that is capable of self-replication, or a cell which is an embryonic stem cell or zygote, etc. The methods and compositions can be employed with cells of a wide variety of vertebrates, including mammals, and especially those of veterinary importance, e.g., canine, feline, equine, bovine, ovine, caprine, rodent such as mouse or rat, lagomorph, swine, etc., in addition to human cell populations.

To the extent that tissue culture of cells may be required, this technique is well known in the art. Freshney (1994) (Culture of Animal Cells, a Manual of Basic Technique, third edition, Wiley-Liss, New York), Kuchler et al. (1977) Biochemical Methods in Cell Culture and Virology, Kuchler, R. J., Dowden, Hutchinson and Ross, Inc., and the references cited therein provides a general guide to the culture of cells. Cultured cell systems often will be in the form of monolayers of cells, although cell suspensions may also be used.

In a preferred embodiment of the invention, the contacting introduces integration of a functional copy of a gene into a chromosome of the subject. In another preferred embodiment, the contacting introduces a DNA fragment of a gene into the nucleus of the cell. In still another preferred embodiment, the contacting introduces an antisense oligonucleic acid into the cell. During any of these methods, the cell may be previously, simultaneously, or subsequently contacted with a cell growth factor.

The compositions and methods of the present invention may be used to deliver to a target cell various antiproliferative factors, such as the proto-oncogene Nov (see, e.g., Martinerie, C. et al., Oncogene 12:1479-1492 (1996)). Proliferation of the smooth muscle cells lining the artery may occur due to injury or to a medical procedure, such as percutaneous transluminal coronary angioplasty (PTCA). Nov expressed in normal artery walls, in response to TGF-β, exerts an antiproliferative effect on vascular smooth muscle cells (VSMCs). Thus, Nov-mediated antiproliferative effects may contribute to maintaining the arterial VSMCs in a fully differentiated state. Emulsion or microemulsion formulations as described herein containing cDNA encoding the Nov proto-oncogene, under the control of a constitutive promoter such as CMV, may be used during PTCA procedures to provide long-term expression of Nov to inhibit smooth muscle cell proliferation during recovery.

In another aspect, the compositions and methods of the present invention may also be used as a delivery vehicle for agents that inhibit induction of the proto-oncogenes c-fos and c-jun. One of the earliest events associated with vascular smooth muscle cell proliferation involves the induction of the c-fos and c-jun genes (see, e.g., Miano, J. M. et al., Am. J. Pathol. 137:761-765 (1990) and Bauters, C. et al., Eur. Heart J. 13:556-559 (1992)). Agents that may be used in a therapeutically effective amount to inhibit expression of the early response genes c-fos and c-jun include without limitation cyclic octapeptide or angiopeptin (i.e. BIM 23014), which is a stable analog of somatostatin (see, e.g., Motomura, N. and Foegh, M. L., Transplant Immunol. 4:99-104 (1996)).

As a further example, the microemulsions described herein may be used as a delivery vehicle for the introduction of small peptides into tumor cells to induce apoptosis (i.e., programmed cell death). For example, the cytotoxic Apo2L (TRAIL) peptide may be used to specifically induce apoptosis in malignant and not normal tissues. Additionally, ectopic expression of the TRID (Trail Receptor without an Intracellular Domain) peptide may be used to protect normal cells from the above-described TRAIL-induced apoptosis in malignant cells (see, e.g., Pan, G. et al., Science 277:815-818 (1997)).

Some methods of gene therapy serve to compensate for a defect in an endogenous gene by integrating a functional copy of the gene into the host chromosome. The inserted gene replicates with the host DNA and is expressed at a level to compensate for the defective gene. Diseases amenable to treatment by this approach are often characterized by recessive mutations. That is, both copies of an endogenious gene must be defective for symptoms to appear. Such diseases include cystic fibrosis, sickle cell anemia, β-thalassemia, phenylketonuria, galactosemia, Wilson's disease, hemochromatosis, severe combined immunodeficiency disease, alpha-1-anititrypsin deficiency, albinism, alkaptonuria, lysosomal storage diseases, Ehlers-Danlos syndrome, hemophilia, glucose-6-phosphate dehydrogenase deficiency, agammaglobulimenia, diabetes insipidus, Lesch-Nyhan syndrome, muscular dystrophy, Wiskott-Aldrich syndrome, Fabry's disease, fragile X-syndrome, and the like. Other recessive mutations are known in the art, and the use of the methods of the present invention to treat them is contemplated herein.

There are several methods for introducing an exogenous functional gene to compensate for the above genetic defects. One approach is the ex vivo introduction of nucleic acids. In this approach, cells are removed from a patient suffering from the disease and contacted with a composition of the present invention in vitro. Cells should be removed from a tissue type in which disease symptoms are manifested. If the cells are capable of replication, and the inventive composition includes a selective marker, cells having internalized and expressed the marker can be selected. However, if selection is not performed, it is particularly desirable that the frequency of gene transfer into cells be high, for example, at least about 1, 5, 10, 25 or 50% of cells. After integration of the nucleic acid into the cell chromosome, and optionally after selecting cells expressing the exogenous nucleic acid, the cells are reintroduced into the patient. In this ex vivo application, and others discussed below (except site-specific recombination to correct dominant mutations), it is not necessary that the gene supplied by the inventive composition be delivered to the same site as is occupied by the defective gene for which it is compensating.

Alternatively, the inventive composition can be introduced directly into a patient in vivo as a pharmaceutical composition. Preferably, the composition should be delivered to the tissue(s) affected by the genetic disorder in a therapeutically effective dose. In this and other methods, a therapeutically effective dose is an amount sufficient to cure, or at least partially arrest, the symptoms of the disease and its complications. Effective doses of the compositions of the present invention, for the treatment of the above described conditions will vary depending upon many different factors, including means of administration, target site, physiological state of the patient, and other medicaments administered. Thus, treatment dosages will need to be titrated to optimize safety and efficacy. Doses ranging from about 10 ng to about 1 g, about 100 ng to about 100 mg, about 1 μg to about 10 mg, or about 30 to about 300 μg DNA per patient are typical.

The compositions of the present invention may also be used to transfect somatic cells such as embryonic stem cells or germline cells such as zygotes to achieve permanent or transient genetic alterations. See, e.g., Jaenisch, Science 240:1468-1474 (1988); Gordon et al. (1984), Methods Enzymol. 101, 414; Hogan et al., Manipulation of the Mouse Embryo. A Laboratory Manual, C.S.H.L. N.Y. ( 1986); and Hammer et al. (1985) Nature 315:680; Gandolli et al. (1987), J. Reprod. Fert. 81:23-28; Rexroad et al. (1988), J. Anim. Sci. 66:947-953 and Eyestone et al. (1989), J. Reprod. Fert. 85:715-720; Camous et al. (1984), J. Reprod. Fert. 72:779-785; Heyman et al. (1987), Theriogenology 27:5968.

As an example, cystic fibrosis (CF) is a usually fatal recessive genetic disease, which has a high incidence in Caucasian populations. The gene responsible for this disease was isolated by Riordan et al., Science 245:1059-1065 (1989). It encodes a protein called the cystic fibrosis transmembrane conductance regulator (CFTR) which is involved in the transfer of chloride ions (C1⁻) through epithelial cell membranes. Mutations in the gene cause defects of C1⁻ secretion in epithelial cells leading to the various clinical manifestations. Although CF has a number of symptoms including thickened exocrine gland secretions, pancreatic deficiency, intestinal blockage and malabsorption of fat, the most serious factor affecting mortality is chronic lung disease. Accordingly, to treat a CF patient, a nucleic acid containing a coding sequence for a functional CFTR gene product can be incorporated into the composition of the invention, and introduced into the patient via nasal administration so that the composition reaches the lungs. The dose of a particulate composition of the invention is preferably about 10⁸-10¹⁰ droplets. See, e.g., U.S. Pat. No. 5,384,128.

As another example, defects in the α or β globin genes (see, e.g., McDonagh and Nienhuis in Hematology of Infancy and Childhood (eds. Nathan & Oski, Saunders, Pa., 1992) at pp. 783-879) can be compensated for by ex vivo treatment of hemopoietic stem cells with a nucleic acid-containing composition of the present invention that includes a functional copy of the α or γ globin gene. The gene integrates into the stem cells in vitro, and the modified cells are then reintroduced into the patient. Defects in the gene responsible for Fanconi Anemia Complement Group C can be treated by an analogous strategy (see, e.g., Walsh et al., J. Clin. Invest. 94:1440-1448 (1994)).

Other applications include the introduction of a functional copy of a tumor suppressor gene into cancerous cells or cells at risk of becoming cancerous. Individuals having defects in one or both copies of an endogenous tumor suppressor gene are particularly at risk of developing cancers. For example, Li-Fraumeni syndrome is a hereditary condition in which individuals receive mutant p53 alleles, resulting in the early onset of various cancers (see, e.g., Harris, Science 262:1980-1981 (1993); Frebourg et al., PNAS 89:6413-6417 (1992); and Malkin et al., Science 250:1233 (1990)). Expression of a tumor suppressor gene in a cancerous cell or a cell at risk of becoming cancerous is effective to prevent, arrest and/or reverse cellular proliferation and other manifestations of the cancerous state. Suitable tumor suppressor genes for use in the invention include without limitation p53 (see, e.g., Buchman et al., Gene 70:245-252 (1988)), APC, DCC, Rb, WT1, and NF1 as described in, e.g., Marx, Science 260:751-752 (1993); and Marshall, Cell 64:313-326 (1991). Compositions of the present invention that incorporate a functional copy of a tumor suppressor gene may be administered, for example, in vivo by the route most proximal to the intended site of action. For instance, skin cancers can be treated by topical administration and leukemia by intravenous administration.

Methods of gene therapy using the compositions of the invention can also be used for prophylactic or therapeutic treatment of patients or their cells that have been infected with or at risk of being infected with, a pathogenic microorganism such as HIV. The effectiveness of antisense nucleic acids in blocking translation, stability, or function of the target gene mRNA, such as impeding virus replication, has been demonstrated in a number of different systems (see, e.g., Friedman et al., Nature 335:452-54 (1988), Malim et al., Cell 58:205-14 (1989) and Trono et al., Cell 59:113-20 (1989)). The plasmid used may include a DNA segment encoding an antisense transcript, which is complementary to a segment of the genome from the pathogenic microorganism. The targeted segment should preferably play an essential role in the lifecycle of the microorganism, and should also be unique to the microorganism or at least absent from the genome of the patient undergoing therapy.

Suitable sites for inhibition on the HIV virus include Tat, Rev or Nef (see, e.g., Chatterjee et al., Science 258:1485-1488 (1992)). Rev is a regulatory RNA binding protein that facilitates the export of unspliced HIV pre mRNA from the nucleus (see, e.g., Malim et al., Nature 338:254 (1989)). Tat is thought to be a transcriptional activator that functions by binding a recognition sequence in 5′ flanking mRNA (see, e.g., Karn et al., Trends Genet. 8:365 (1992)). The nucleic acid-containing composition of the invention may be introduced to leukocytes or hemopoietic stem cells, either in vitro or by intravenous injection in a therapeutically effective dose. The treatment can be administered prophylactically to HIV persons, or to persons already infected with HIV.

Analogous methods are used for suppressing expression of endogenous recipient cell genes encoding adhesion proteins. Suppression of adhesion protein expression is useful in aborting undesirable inflammatory responses. Adhesion proteins that can be suppressed by antisense segments present in selected vectors include integrins, selectins, and immunoglobulin (Ig) superfamily members (see, e.g., Springer, Nature 346:425-433 (1990); Osborn, Cell 62:3 (1990); and Hynes, Cell 69:11 (1992)). Integrins are heterodimeric transmembrane glycoproteins consisting of an α chain (120-180 kDa) and a β chain (90-110 kDa), generally having short cytoplasmic domains. The three known integrins, LFA-1, Mac-1 and P150,95, have different alpha subunits, designated CD11a, CD11b and CD11c, and a common beta subunit designated CD18. LFA-1 (a₁β₂) is expressed on lymphocytes, granulocyte and monocytes, and binds predominantly to an Ig-family member counter-receptor termed ICAM-1 (and perhaps to a lesser extent ICAM-2). ICAM-1 is expressed on many cells, including leukocytes and endothelial cells, and is up-regulated on vascular endothelium by cytokines such as TNF and IL-1. Mac-1 (α_{M}β₂) is distributed on neutrophils and monocytes, and also binds to ICAM-1 (and possibly ICAM-2). The third β2 integrin, P150,95 (α_{X}β₂), is also found on neutrophils and monocytes. The selectins consist of L-selectin, E-selectin and P-selectin.

Gene therapy relies on the efficient delivery of therapeutic genes to target cells. Most of the somatic cells that have been targeted for gene therapy, e.g., hematopoietic cells, skin fibroblasts and keratinocytes, hepatocytes, endothelial cells, muscle cells and lymphocytes, are normally non-dividing. Retroviral vectors, which are the most widely used vectors for gene therapy, unfortunately require cell division for effective transduction (see, e.g., Miller et al., Mol. Cell. Biol. 10:4239-4242 (1990)). The same is true for other gene therapy vectors such as the adeno-associated vectors (see, e.g., Russell et al., P.N.A.S. USA 91:8915-8919 (1994); Alexander et al., J. Virol. 68:8282-8287 (1994); and Srivastrava, Blood Cells 20:531-538 (1994)).

Recently, HIV-based vectors have been reported to transfect non-dividing cells. Nonetheless, the majority of stem cells, a preferred target for many gene therapy treatments, are normally not proliferating. Thus, the efficiency of transduction is often relatively low, and the gene product may not be expressed in therapeutically or prophylactically effective amounts. This has led investigators to develop techniques such as stimulating the stem cells to proliferate prior to or during gene transfer (e.g.,,by treatment with growth factors) pretreatment with 5-fluorouracil, infection in the presence of cytokines, and extending the vector infection period to increase the likelihood that stem cells are dividing during infection. Any of these techniques may be used in the present invention.

In the above compositions and methods of their use, the bioactive agent, nucleic acid, or hydrophobically-modified nucleic acid may be a nucleic acid vaccine or a hydrophobically-modified nucleic acid vaccine. In preferred embodiments, the nucleic acid vaccine is a DNA vaccine. In further preferred embodiments, the nucleic acid vaccine or DNA vaccine is capable of, and in the inventive methods actually does, induce at least one of a cytotoxic T-lymphocyte (CTL) response, a T-helper response, and a neutralizing antibody response in a subject to whom the composition is administered. In another preferred embodiment, the nucleic acid vaccine or DNA vaccine is capable of, and in the inventive methods actually does, potentiate at least one of a pre-existing CTL response to an antigen, a pre-existing T-helper response to an antigen, and a pre-existing neutralizing antibody response to an antigen, in a subject to whom the composition is administered.

After a given cell is contacted with a nucleic acid construct that encodes a gene of interest, it is often useful to detect which cells or cell lines express the gene product and to assess the level of expression of the gene product in engineered cells. This requires the detection of nucleic acids that encode the gene products.

Nucleic acids and proteins are detected and quantified herein by any of a number of means well known to those of skill in the art. The detection of proteins may be accomplished by analytic biochemical methods such as spectrophotometry, radiography, electrophoresis, capillary electrophoresis, high performance liquid chromatography (HPLC), thin-layer chromatography (TLC), hyperdiffusion chromatography, and the like, and various immunological methods such as fluid or gel precipitin reactions, western analysis, immunodiffusion (single or double), immunoelectrophoresis, radioimmunoassays (RIAs), enzyme-linked immunosorbent assays (ELISAs), immunofluorescent assays, and the like. The detection of nucleic acids proceeds by well-known methods such as Southern analysis, northern analysis, gel electrophoresis, seqencing, primer extension, PCR, radiolabeling, scintillation counting, affinity chromatography, and the like.

The selection of a nucleic acid hybridization format is not critical. A variety of nucleic acid hybridization formats are known to those skilled in the art. For example, common formats include sandwich assays and competition or displacement assays. Hybridization techniques are generally described in “Nucleic Acid Hybridization, A Practical Approach,” Ed. Hames, B. D. and Higgins, S. J., IRL Press, 1985.

The sensitivity of the hybridization assays may be enhanced through use of a nucleic acid amplification system which multiplies the target nucleic acid being detected. In vitro amplification techniques suitable for amplifying sequences for use as molecular probes or for generating nucleic acid fragments for subsequent subcloning are known. Examples of techniques sufficient to direct persons of skill through such in vitro amplification methods, including the polymerase chain reaction (PCR) the ligase chain reaction (LCR), QB-replicase amplification and other RNA polymerase mediated techniques (e.g., NASBA) are found in Berger, Sambrook, and Ausubel, as well as Mullis et al. (1987), U.S. Pat. No. 4,683,202; PCR Protocols: A Guide to Methods and Applications (Innis et al. eds) Academic Press Inc., San Diego, Calif. (1990) (Innis); Arnheim & Levinson (Oct. 1, 1990), C&EN 36-47; The Journal of NIH Research (1991), 3:81-94; Kwoh et al. (1989), Proc. Natl. Acad. Sci. USA 86:1173; Guatelli et al. (1990), Proc. Natl. Acad. Sci. USA 87:1874; Lomell et al. (1989), J Clin. Chem., 35:1826; Landegren et al. (1988), Science 241:1077-1080; Van Brunt (1990), Biotechnology 8:291-294; Wu and Wallace (1989), Gene 4:560; Barringer et al. (1990), Gene 89:117, and Sooknanan and Malek (1995), Biotechnology 13:563-564. Improved methods of cloning in vitro amplified nucleic acids are described in Wallace et al., U.S. Pat. No. 5,426,039. Other methods recently described in the art are the nucleic acid sequence based amplification (NASBA™, Cangene, Mississauga, Ontario) and Qβ Replicase systems. These systems can be used to directly identify mutants where the PCR or LCR primers are designed to be extended or ligated only when a select sequence is present. Alternatively, the select sequences can be generally amplified using, for example, nonspecific PCR primers and the amplified target region later probed for a specific sequence indicative of a mutation.

Another suitable method to enhance the sensitivity of detecting delivery of nucleic acid to cells by the compositions of the present invention is direct in situ PCR (IS-PCR). During IS-PCR, a label is incorporated directly into the amplicon throughout the PCR process. For example, hapten-labeled nucleotide analogs such as biotin-11-dUTP, biotin-14-dATP, digoxigenin-11-dUTP or fluorescein-15-dATP may be added to the PCR mixture. The incorporated hapten can subsequently be detected in the amplicon by enzyme-conjugated antibodies and chromogenic substrates. Direct label incorporation results in the labeling of all nucleic acids synthesized during the PCR process, resulting in high sensitivity with the detection of single copy genes being possible.

Additionally, delivery of nucleic acid to cells by the compositions of the invention may be detected by indirect IS-PCR. In indirect IS-PCR, a standard IS-PCR mixture is used that consists of TaqI DNA polymerase, buffer, Mg++, dNTPs, and appropriate PCR primer pairs. Following PCR, the specimen is fixed briefly to maintain the localization of PCR product, and is then detected by subsequent hybridization with a suitable labeled probe. Specific hybridization probes may be labeled oligonucleotides, labeled PCR product, or labeled genomic sequences. Indirect IS-PCR overcomes the problem associated with specificity because the probe only hybridizes to the target specific amplicon sequences produced during the PCR, and not to DNA produced by nonspecific synthesis.

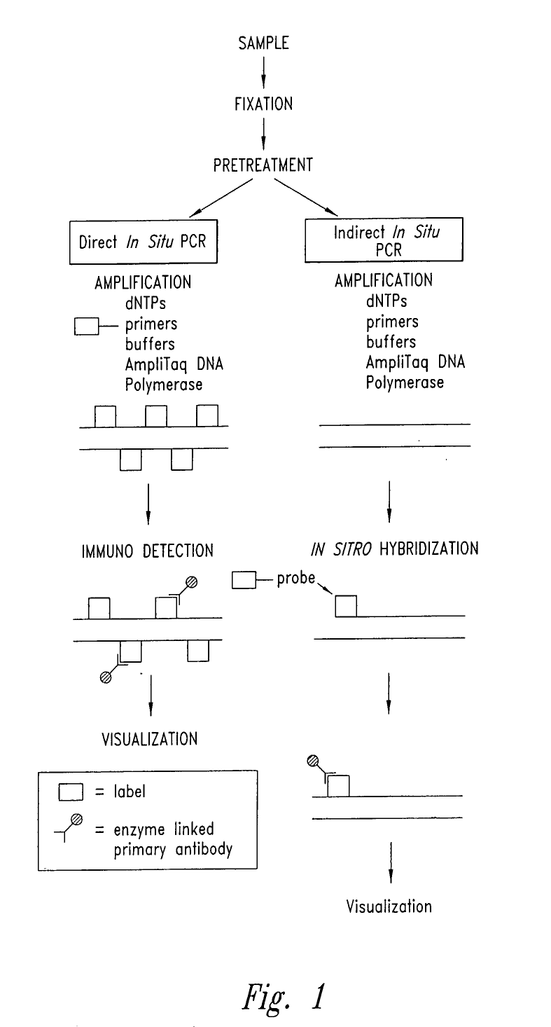

Both direct and indirect IS-PCR procedures are schematically illustrated in FIG. 1.

The expression of the gene of interest may be detected or quantified by a variety of methods. The expression of a gene may be measured by monitoring RNA or protein levels. Preferred methods involve the use of specific antibodies or specific nucleic acid probes.

Methods of producing polyclonal and monoclonal antibodies are known to those of skill in the art. See, e.g., Coligan (1991), Current Protocols in Immunology, Wiley/Greene, NY; and Harlow and Lane (1989), Antibodies: A Laboratory Manual, Cold Spring Harbor Press, NY; Stites et al. (eds.) Basic and Clinical Immunology (4th ed.) Lange Medical Publications, Los Altos, Calif., and references cited therein; Goding (1986), Monoclonal Antibodies. Principles and Practice (2d ed.) Academic Press, New York, N.Y.; and Kohler and Milstein (1975), Nature 256:495-497. Such techniques include antibody preparation by selection of antibodies from libraries of recombinant antibodies in phage or similar vectors. See, Huse et al. (1989), Science 246:1275-1281; and Ward et al. (1989), Nature 341:544-546. Specific monoclonal and polyclonal antibodies and antisera will usually bind with a K_{D} of at least about 0.1 mM, more usually at least about 1 μM, preferably at least about 0.1 μM or better, and most typically and preferably, 0.01 μM or better.

The presence of a desired polypeptide (including peptide, transcript, or enzymatic digestion product) in a sample may be detected and quantified using western blot analysis. The technique generally comprises separating sample products by gel electrophoresis on the basis of molecular weight, transferring the separated proteins to a suitable solid support (such as a nitrocellulose filter, a nylon filter, or derivatized nylon filter), and incubating the sample with labeling antibodies that specifically bind to the analyte protein. The labeling antibodies specifically bind to analyte on the solid support. These antibodies are directly labeled, or alternatively are subsequently detected using labeling agents such as antibodies (e.g., labeled sheep anti-mouse antibodies where the antibody to an analyte is a murine antibody) that specifically bind to the labeling antibody.

An alternative means for determining the level of expression of the gene is in situ hybridization. In situ hybridization assays are well known and are generally described in Angerer et al. (1987), Methods Enzymol. 152:649-660. In an in situ hybridization assay cells are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes, fluorescent, or chemiluminescent reporters.

An important aspect of this invention is the use of the delivery vehicles provided herein to introduce selected genes into cells in vitro, in vivo, ex vivo or in situ, followed by expression of the selected gene in the host cell. Thus, the nucleic acid-component of the inventive composition specifically encompass genetic sequences that are capable of being expressed in a host cell. Promoter, enhancer, stress or chemically-regulated promoters, antibiotic-sensitive or nutrient-sensitive regions, as well as therapeutic protein encoding sequences, may be included as required.

For instance, the expression of various clinically relevant cDNAs may be accomplished through the use of tissue-specific promoters. Targeted gene expression for the treatment of various cancers may be accomplished through the use of tissue-specific (e.g., breast-, prostate-, and melanoma-specific) promoters and disease-specific promotors (e.g., carcinoembryonic antigen, HER-2/neu, Myc-Max response elements, DF3/MUC, etc.). Gene expression may also be targeted at conditions specific to the tumour microenvironment, such as glucose deprivation and hypoxia. As an example, it is known chronic hypoxia occurs in tissues which are more than 100-200 microns away from a functional blood supply. In solid tumours, hypoxia is widespread as a result of the prolific nature of tumour cells as well as the fact that during tumour growth newly formed blood supply is disorganized. Therefore, compositions and methods of the present invention may employ hypoxia response elements (HRE) derived from the oxygen-regulated phosphoglycerate kinase gene to control gene expression in human tumour cells. See, e.g., Dachs, G. U. et al. “Targeting Gene Therapy to Cancer: A Review” Oncol. Res. 9:313-325, 1997).

In brief summary, the expression of natural or synthetic nucleic acids is typically achieved by operably linking a nucleic acid of interest to a promoter (which is either constitutive or inducible), incorporating the construct into an expression vector, and introducing the vector into a suitable host cell. Typical vectors contain transcription and translation terminators, transcription and translation initiation sequences, and promoters useful for regulation of the expression of the particular nucleic acid. The vectors optionally comprise generic expression cassettes containing at least one independent terminator sequence, sequences permitting replication of the cassette in eukaryotes, or prokaryotes, or both (e.g.; shuttle vectors) and selection markers for both prokaryotic and eukaryotic systems. Vectors are suitable for replication and integration in prokaryotes, eukaryotes, or preferably both. See, Giliman and Smith (1979), Gene 8:81-97; Roberts et al. (1987), Nature 328:731-734; Berger and Kimmel, Guide to Molecular Cloning Techniques, Methods in Enzymology, Volume 152, Academic Press, Inc., San Diego, Calif. (Berger); Sambrook et al. (1989), Molecular Cloning-A Laboratory Manual (2nd ed.), Vol. 1-3, Cold Spring Harbor Laboratory, Cold Spring Harbor Press, N.Y. (Sambrook); and F. M. Ausubel et al., Current Protocols in Molecular Biology, eds., Current Protocols, a joint venture between Greene Publishing Associates, Inc. and John Wiley & Sons, Inc. (1994 Supplement) (Ausubel). Product information from manufacturers of biological reagents and experimental equipment also provide information useful in known biological methods. Such manufacturers include the SIGMA chemical company (St. Louis, Mo.), R&D Systems (Minneapolis, Minn.), Pharmacia LKB Biotechnology (Piscataway, N.J.), Clontech Laboratories, Inc. (Palo Alto, Calif.), Chem Genes Corp., Aldrich Chemical Company (Milwaukee, Wis.), Glen Research, Inc., Gibco BRL Life Technologies, Inc. (Gaithersburg, Md.), Fluka Chemica-Biochemika Analytika (Fluke Chemie AG, Buchs, Switzerland), and Applied Biosystems (Foster City, Calif.), as well as many other commercial sources known to one of skill.

In addition to a promoter sequence, the expression cassette should also contain a transcription termination region downstream of the structural gene to provide for efficient termination. The termination region may be obtained from the same source as the promoter sequence or may be obtained from a different source.

If the mRNA encoded by the selected structural gene is to be efficiently translated, polyadenylation sequences are also commonly added to the vector construct. Two distinct sequence elements are required for accurate and efficient polyadenylation: GU or U rich sequences located downstream from the highly conserved polyadenylation site. Termination and polyadenylation signals that are suitable for the present invention include those derived from, for example, SV40, CMV, from any partial genomic copy of a gene or a combination thereof.

Selected genes are normally expressed when the DNA sequence is functionally inserted into a vector. “Functionally inserted” means that it is inserted in proper reading frame and orientation and operably linked to appropriate regulatory elements. Typically, a gene will be inserted downstream from a promoter and will be followed by a stop codon, although production as a hybrid protein, followed by cleavage may be used if desired.

Expression vectors containing regulatory elements from eukaryotic viruses such as retroviruses are typically used. SV40 vectors include pSVT7 and pMT2. Vectors derived from bovine papilloma virus include pBV-1MTHA, and vectors derived from Epstein Bar virus include pHEBO and p2O5. Other exemplary vectors include pMSG, pA v009/A⁺, pMTO10/A⁺, pMAMneo-5, baculovirus pDSVE, and any other vector allowing expression of proteins under the direction of the SV-40 early promoter, SV-40 late promoter, metallothionein promoter, murine mammary tumor virus promoter, Rous sarcoma virus promoter, polyhedrin promoter, or other promoters shown effective for expression in eukaryotic cells.

While a variety of vectors may be used, it should be noted that viral vectors such as retroviral vectors are useful for modifying eukaryotic cells because of the high efficiency with which the retroviral vectors transfect target cells and integrate into the target cell genome. Additionally, the retroviruses harboring the retroviral vector are capable of infecting cells from a wide variety of tissues.

In addition to the retroviral vectors mentioned above, cells may be lipofected with adeno-associated viral vectors. See, e.g., Methods in Enzymology, Vol. 185, Academic Press, Inc., San Diego, Calif. (D. V. Goeddel, ed.) (1990) or M. Krieger (1990), Gene Transfer and Expression—A Laboratory Manual, Stockton Press, New York, N.Y., and the references cited therein. Adeno-associated viruses (AAVs) require helper viruses such as adenovirus or herpes virus to achieve productive infection. In the absence of helper virus functions, AAV integrates (site-specifically) into a host cell's genome, but the integrated AAV genome has no pathogenic effect. The integration step allows the AAV genome to remain genetically intact until the host is exposed to the appropriate environmental conditions (e.g., a lytic helper virus), whereupon it re-enters the lytic life-cycle. Samulski (1993), Current Opinion in Genetic and Development 3:74-80, and the references cited therein provide an overview of the AAV life cycle. See also West et al. (1987), Virology 160:38-47; Carter et al. (1989), U.S. Pat. No. 4,797,368; Carter et al. (1993), WO 93/24641; Kotin (1994), Human Gene Therapy 5:793-801; Muzyczka (1994), J. Clin. Invest. 94:1351 and Samulski, supra, for an overview of AAV vectors.

Plasmids designed for producing recombinant vaccinia, such as pGS62 (Langford, C. L. et al. (1986), Mol. Cell. Biol. 6:3191-3199) may also be used. This plasmid consists of a cloning site for insertion of foreign nucleic acids, the P7.5 promoter of vaccinia to direct synthesis of the inserted nucleic acid, and the vaccinia TK gene flanking both ends of the foreign nucleic acid.

Whatever vector is used, generally the vector is genetically engineered to contain, in expressible form, a gene of interest. The particular gene selected will depend on the intended treatment.

The vectors further usually comprise selectable markers which result in nucleic acid amplification such as the sodium, potassium ATPase, thymidine kinase, aminoglycoside phosphotransferase, hygromycin B phosphotransferase, xanthine-guanine phosphoribosyl transferase, CAD (carbamyl phosphate synthetase, aspartate transcarbamylase, and dihydroorotase), adenosine deaminase, dihydrofolate reductase, and asparagine synthetase and ouabain selection. Alternatively, high yield expression systems not involving nucleic acid amplification are also suitable, such as using a bacculovirus vector in insect cells, with the encoding sequence under the direction of the polyhedrin promoter or usher strong baculovirus promoters.

When nucleic acids other than plasmids are used the nucleic acids can contain nucleic acid analogs, for example, the antisense derivatives described in a review by Stein et al., Science 261:1004-1011 (1993) and in U.S. Pat. Nos. 5,264,423 and 5,276,019.

The compositions of the invention can be used to achieve regulation of antigens useful as cancer vaccines. Genes specifying antigens expressed on the surface of tumor cells has been the subject of extensive research over the past several years. As an example, antigens associated with malignant melanoma have been described. Expression of such tumor antigens in muscle cells using the compositions of the present invention would result in T/B-cell mediated immune response (i.e., vaccination) against tumors bearing the activating antigen. Such DNA vaccines may have merit for treatment of various cancers including various colorectal, lung, and breast carcinomas.

The following examples are provided for purposes of illustration, not limitation.

### EXAMPLES

### Example 1

A stock mixture of 29.66 g of Caprol 10G4O (HLB 6.2, Abitec Corp., Columbus, Ohio) and 70.41 g of Cremophor EL (HLB 12-14, BASF Corp., Ludwigshafen, Germany) was prepared and labeled I362-19-01. This mixture was 29.64 wt % Caprol and 70.36 wt % Cremophor. A mixture of 3.354 g of Corn Oil (Spectrum Chemical, Gardena, Calif.) and 6.00 g of I362-19-01 was then prepared and labeled I362-27-01. This mixture had the following composition.

| | |
|---|---|
| Cremophor EL | 45.14 wt % |
| Caprol 10G4O | 19.03 wt % |
| Corn Oil | 35.83 wt % |

Preparation of the formulation

To prepare I362-32-01, 0.908 g of I362-27-01 were weighed into a test tube. Prior to use the I362-27-01 mixture was vortexed to ensure homogeneity and that no stratification of the mixture had occurred on standing. The solution of plasmid pbetagal Control Vector (lacZ, Clonetech, Palo Alto, Calif.), used at a concentration of approximately 4 mg/mL, was also vortexed prior to use and delivered by calibrated electronic pipette to the I362-27-01 mixture. The density of the solution was assumed to be 1 g/mL. Therefore, 0.100 g of pbetagal were added to the mixture I362-32-01 previously weighed out to give a sample composed of the following.

| | | |
|---|---|---|
| Cremophor EL | 40.66 | wt % |
| Caprol 10G4O | 17.15 | wt % |
| Corn Oil | 32.27 | wt % |
| pbetagal solution | 9.92 | wt % |

The resulting mixture was vortexed for about 30 seconds to mix the components.

### Example 2

I362-27-01 was used as the surfactant/co-surfactant/oil mixture.

Sample I362-32-02 was prepared as described above for sample I362-32-01 with the following exceptions. The pbetagal solution (0.400 g of a 4 mg/mL pbetagal solution) was mixed with 0.200 g of sterile water (Fisher Scientific, Springfield, N.J.). This diluted pbetagal solution was the added to 0.407 g of I362-27-01 to give a mixture of the following composition.

| | | |
|---|---|---|
| Cremophor EL | 18.24 | wt % |
| Caprol 10G4O | 7.69 | wt % |
| Corn Oil | 14.49 | wt % |
| pbetagal solution | 39.72 | wt % |
| Sterile water | 19.86 | wt % |

The resulting mixture was vortexed for about 30 seconds to mix the components.

### Example 3

I362-27-01 was used as the surfactant/co-surfactant/oil mixture.

Sample I362-34-01 was prepared as described above for sample I362-32-01 with the following exceptions. To 0.922 g of I362-27-01 was added 0.102 g of a Calf Thymus DNA (genomic)/ethidium bromide solution (CT/EB). The concentration of the CT/EB solution was 400 μg/mL. The CT/EB solution was assumed to be 1 g/mL and was delivered via calibrated electronic pipette. The resulting mixture had the following composition.

| | |
|---|---|
| Cremophor EL | 40.63 wt % |
| Caprol 10G4O | 17.12 wt % |
| Corn Oil | 32.25 wt % |
| CT/EB solution | 10.00 wt % |

The resulting mixture was vortexed for about 30 seconds to mix the components.

### Example 4

I362-27-01 was used as the surfactant/co-surfactant/oil mixture.

Sample I362-34-02 was prepared as described above for sample I362-32-01 with the following exceptions. To 0.405 g of I362-27-01 was added 0.607 g CT/EB from a 400 μg/mL CT/EB solution. The CT/EB solution was delivered via a calibrated electronic pipette. The resulting mixture had the following composition.

| | | |
|---|---|---|
| Cremophor EL | 18.06 | wt % |
| Caprol 10G4O | 7.61 | wt % |
| Corn Oil | 14.33 | wt % |
| CT/EB solution | 60.00 | wt % |

The resulting mixture was vortexed for about 30 seconds to mix the components.

### Example 5

Preparation of the surfactant/co-surfactant/oil mixture.

To 7.064 g of Cremophor EL were added 3.103 g of Caprol 10G4O. Corn Oil (0.325 g) was added to 0.573 g of the Cremophor/Caprol mixture. The mixture was warmed at 50° C. and vortexed intermittently until the components were mixed.

Preparation of the formulation.

A solution of plasmid pbetagal at a concentration of approximately 6.5 μg/μL was combined with the above surfactant/co-surfactant/oil mixture and vortexed for about 30 seconds to thoroughly mix the components. A formulation of the following composition resulted.

| | |
|---|---|
| Cremophor EL | 40.01 wt % |
| Caprol 10G4O | 17.06 wt % |
| Corn Oil | 32.37 wt % |
| pbetagal solution | 10.56 wt % |

### Example 6

The formulation was prepared as follows. A solution containing the β-galactosidase control plasmid, pNX1100-5, at a concentration of approximately 4.3 mg/mL in TE buffer (10 mM Tris, 0.1 mM EDTA, pH 7.5), was placed in a syringe. Corn oil (1.407 g), Cremophor EL (1.751 g), and Caprol 10G4O (0.758 g) were weighed into the syringe in the order listed below. Using a Luer-lok connector, another syringe was connected and the contents were pushed back and forth between the syringes about 20 to 25 times to mix them. The resulting mixture having the following composition was placed in a water bath at 55° C. to warm for 2 hours.

| | |
|---|---|
| pNX1100-5 solution | 10.49 wt % |
| Corn Oil | 32.16 wt % |
| Cremophor EL | 40.02 wt % |
| Caprol 10G4O | 17.33 wt % |

### Example 7

Cremophor EL and Caprol 10G4O were weighed into the syringe as a mixture which was 70.36 wt % Cremophor and 29.64 wt % Caprol. Corn oil was also weighed into the syringe. The aqueous portion of the formulation has an 1844-base pair portion of the lacZ gene (an EcoRV/NdeI fragment) labeled with digoxigenin in TE at approximately 40 μg/mL, which was added to the syringe last. The mixture was pushed between two syringes to mix as previously described and placed in a water bath at 55° C. to warm for 2 hours. The resulting formulation had the following composition.

| | | |
|---|---|---|
| Cremophor EL | 0.8218 g | 40.20 wt % |
| Caprol 10G4O | 0.3462 g | 16.94 wt % |
| Corn Oil | 0.6700 g | 32.78 wt % |
| DIG-lacZ solution | 0.2060 g | 10.08 wt % |

### Example 8

The formulation components were weighed into a glass vial in the amounts and order listed below. The Cremophor and Caprol were added as a mixture as described above. The resulting mixture was vortexed for a few seconds then placed in a water bath at 60° C. for one hour.

| | | | |
|---|---|---|---|
| Corn Oil | 0.3010 g | 14.97 | wt % |
| Cremophor EL | 0.3729 g | 18.55 | wt % |
| Caprol 10G4O | 0.1571 g | 7.82 | wt % |
| DIG-lacZ solution | 0.2100 g | 10.45 | wt % |
| Sterile Water | 0.9690 g | 48.21 | wt % |

### Example 9

The following components were weighed into a syringe in the amounts and in the order listed below. The Cremophor and Caprol were added as a mixture as described above. The aqueous phase was TE buffer containing fluorescein phalloidin. The TE buffer containing the fluorescein phalloidin was prepared by removing 500 μL of a 200 Units/mL fluorescein phalloidin solution in methanol and evaporating to dryness. The appropriate amount of TE buffer was added to the dried fluorescein phalloidin to redissolve it.

| | | |
|---|---|---|
| Corn Oil | 0.992 g | 32.82 wt % |
| Cremophor EL | 1.209 g | 40.00 wt % |
| Caprol 10G4O | 0.311 g | 16.86 wt % |
| Phalloidin/TE buffer | 0.312 g | 10.32 wt % |

After the components were combined in the syringe, they were mixed back and forth between two syringes as previously described. The formulation was then warmed in a water bath at 55° C. for 2 hours.

### Example 10

Sample J038-42-02 was prepared in the same manner as J038-42-01 with the exception that more TE buffer was added. The fluorescein phalloidin was prepared as described above. The components of the formulation were and in the order and in the amounts listed below.

| | | |
|---|---|---|
| Corn Oil | 0.593 g | 14.85 wt % |
| Cremophor EL | 0.737 g | 18.47 wt % |
| Caprol 10G4O | 0.311 g | 7.78 wt % |
| Phalloidin/TE buffer | 2.352 g | 58.90 wt % |

### Example 11

³H-THYMIDINE ASSAY—MICROEMULSION TOXICITY

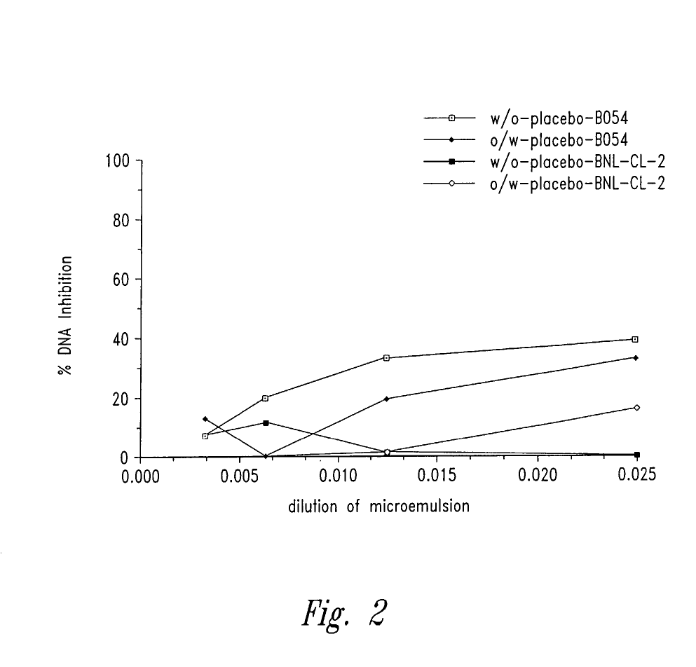

In order to determine if the formulated microemulsions inhibited de novo DNA synthesis to significant levels (i.e., >50% inhibition) baboon smooth muscle cells (BO54) or mouse liver cells (BNL-CL-2) were grown in DMEM/F12 or DMEM, respectively, containing 10% fetal bovine serum in 24-well microtiter plates. Following growth to approximately 80% confluency, placebo microemulsions formulated in either a 10% (i.e., oil-in-water emulsion: O/W) or 60% (i.e., water-in-oil: W/O) aqueous environment were added to individual wells at various dilutions, and incubated for an additional 3 hours at 37° C. and a 5% CO₂ humidified atmosphere. Media was aspirated from wells, and 1 ml fresh media containing 1 μCi ³H-thymidine was added to each well, and incubated overnight at 37° C. and a 5% CO₂ humidified atmosphere. The media was aspirated and the cells were washed twice with 5% cold TCA, and subsequently lysed with 250 μl 0.2N NaOH. Supernatants were removed and radioactive counts per minute was determined in a Packard liquid scintillation counter. All data was compared against control wells receiving no microemulsion. As illustrated in the graph of FIG. 2, both cell lines were inhibited by less than 50% at a dilution of 1/40. Additionally, DNA synthesis in liver cells was only marginally affected by either the O/W or W/O microemulsions.

### Example 12

TRANSFECTION OF MICROEMULSIONS CONTAINING DIGOXIGENIN-LABELED DNA

In an effort to determine if transfection could be enhanced by attachment of a cholesterol (lipid) groups to the DNA, we excised an EcoRV/NdeI fragment from the lacZ region of the vector pNX1100-5, and uniformly labeled the fragment with digoxigenin according to established procedures (Boehringer Mannheim, Indianapolis, Ind., and see, e.g., Schmitz, G. G. et al., Anal. Biochem. 192:222-231 (1991)). Baboon smooth muscle cells (B054) or colon carcinoma cells (SWI-222) were grown to approximately 80% confluency in 96-well microtiter plates. Subsequently, either W/O or O/W microemulsions containing the digoxigenin labeled lacZ fragment were added to the cells in DMEM-10% FBS media. Following a 3 hour incubation at 37° C. and a 5% CO₂ humidified atmosphere, the media was aspirated and fresh DMEM-10% FBS media was added. After an additional incubation overnight as above, the media was aspirated, the cells were washed in Hanks buffered saline solution (HBSS), and then fixed in 10% buffered formalin for 8 hours at 4° C. The cells were washed with HBSS and then mouse anti-digoxigenin IgG₁ antibody (0.1 μg/mL)in chicken serum PBS Tween buffer was added. After incubation overnight at 4° C., the cells were washed in PBS-Tween and then goat anti-mouse IgG₁ biotin (1 μg/mL) was added for 2 hours at 4° C. The cells were washed in PBS-Tween and then streptavidin-horseradish peroxidase (SA-HRP) was added for 1 hour at 4° C. The cells were washed again, then 2,2′-azinobis(3-ethylbenz thiazoline sulfonic acid), diammonium salt (ABTS, Sigma, St. Louis, Mo.) was added, and allowed to react at room temperature for 30 minutes. The colorometric results were measured at an OD=415-490 nm on a Microplate EL310 autoreader (BIO-TEK Instruments).

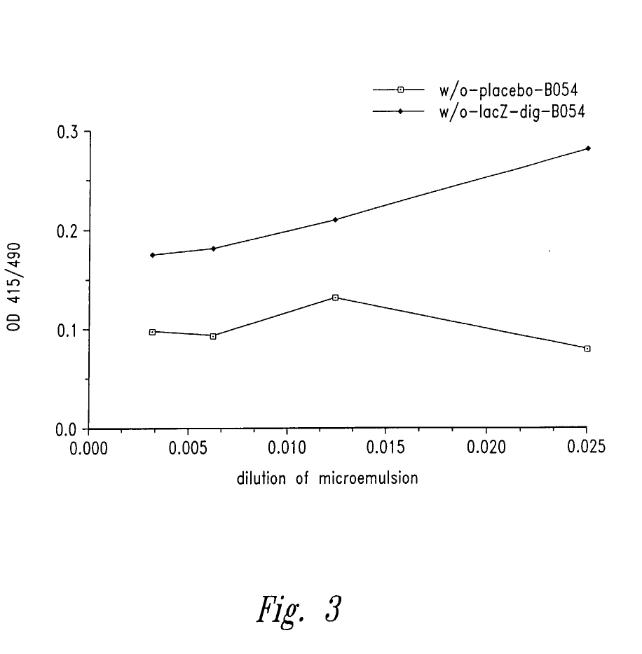

As illustrated in the graph of FIG. 3, a nearly linear increase in the OD_{415/490} signal was observed with increasing levels of the W/O microemulsion containing digoxigenin-lacZ fragment. Similar levels of transfection were observed with the O/W microemulsion containing digoxigenin-lacZ fragment (data not shown).

### Example 13

PEPSIN DIGESTION

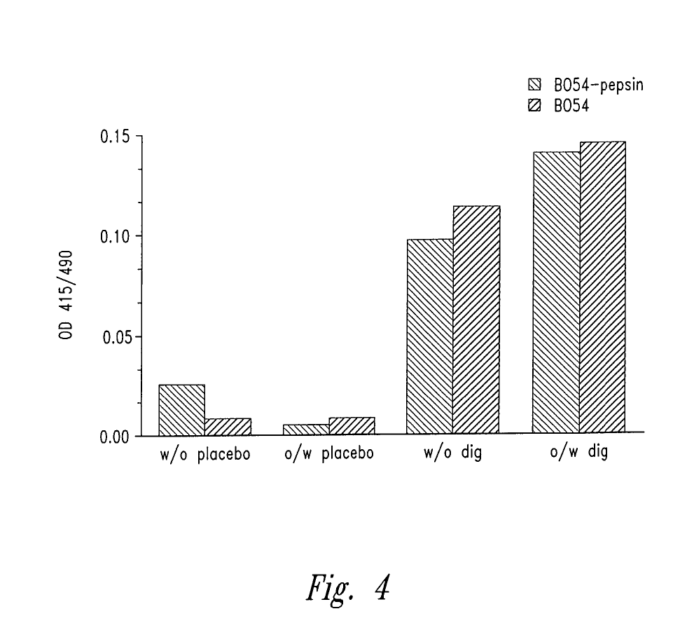

The internalization of the transfected digoxigenin-lacZ fragment in both W/O and O/W microemulsions has been confirmed by treatment of transfected B054 baboon smooth muscle cells with pepsin. Duplicate sets of B054 cells were transfected with either W/O or O/W microemulsions containing the digoxigenin-lacZ fragment as described above. One set of transfected cells was treated with 0.01% Pepsin/0.2 N HCl and washed, then incubated overnight with mouse anti-digoxigenin IgG₁ antibody at 4° C. The presence of digoxigenin in transfected tissue was detected by subsequently incubating with SA-HRP as described in Example 12. Because pepsin is known to strip the cell membranes off intact cells, it can serve as an indirect assay for internalized as opposed to membrane bound molecules. Cells treated with pepsin retained the transfected lacZ fragment at levels that were statistically indistinguishable from the non-pepsin treated controls, as shown in FIG. 4. A slightly higher level of internalized lacZ fragment was observed following transfection with the O/W formulation.

### Example 14

ASSAY TO CHARACTERIZE CELLULAR UPTAKE BY VIABLE CELLS

General: This is a tissue culture assay that screens lipid emulsions for their ability to enter cells and characterizes the method of their entry. The evaluation of the emulsion treated tissue culture cells by transmission electron microscopy indicates whether the emulsion droplets enter by chemical microporulation, endocytosis, or phagocytosis. Moreover, stability can be evaluated by monitoring whether droplets penetrate intracellular membranes such as the nucleus, and cytoplasmic organelles such as mitochondria, Golgi apparatus vesicles, smooth and rough endoplasmic reticulum, lysosomes, phagocytic vacuoles, etc. Compositions of the present invention may penetrate the cell, nuclear and organelle membranes by microporulation. In contrast, the cell entry mechanisms of drug delivery systems that rely on cationic lipid, such as liposomes, lipofectamine, polymer microparticles, lipid particles such as Lipoplexes and complex molecules to which nucleic acids are bound, all of which enter the cells by either fusion or more commonly by endocytosis. These entry mechanisms ultimately deposit the nucleic acid into the cell cytoplasm and, therefore, are unable to effectively penetrate the nuclear or organelle membranes because there is no direct nucleic acid delivery to these intracellular sites.

Methods: Two chamber tissue culture slides (Nunc, Inc.) are seeded with explanted non-human primate artery smooth muscle cells 24 hrs prior to the emulsion testing. This allows the cells to adhere to the glass surface and grow to 80% confluency prior to applying the test emulsion. The test emulsions of the invention are diluted 1:20 with tissue culture media. One chamber of the two chambered slides remains as a untreated control while the other chamber receives the test emulsion. The exposure and recovery time intervals can be varied for different experiments; however, the standard preferred method is incubating the cells with the diluted test emulsion for 2 hours, followed by washing the cells and then replacing the media with fresh media, followed by a 24 hour recovery period. All time points (exposures and recovery) are done in duplicate. At the end of the exposure and recovery periods, the media is aspirated from the cells and the plates flooded with Kamiovsky's (formaldehyde-glutaraldelhyde) electron microscopy fixative overnight and then stored in cacodylate buffer at 4° C. (see, e.g., Karnovsky, M. J., J. Cell Biol. 27:137A (1965); Bennett, H. S. and Luft, J. H., J. Biophys. Biochem. Cytol. 6:113 (1959); and Palade, B. E. Proc. 3rd Int. Congr. Electron Microscopy, London, p. 129 (1956)).

The fixed cells are embedded at the end of an epoxy resin cylinder, directly on the glass slide from the bottom of the culture chamber. The cured epoxy cylinder and attached glass slide are dipped in liquid nitrogen and the glass slide fractured away from the monolayer of epoxy resin impregnated cells at the end of the resin cylinder. The epoxy cylinder is trimmed and sectioned with a diamond knife at 80-100 nm, applied to a copper grid, processed and stained for examination with a model 100-SX, JEOL transmission electron microscope (see, e.g., Gallucci, B. B., Sale, G. E., McDonald, G. B., Epstein, R., Shulman, H. M. and Thomas, E. D. (1982), The Am. J. of Surgical Pathology 6(4):293-304; Oda, D., Lee, S. P. and Hayashi, A. (1991), Laboratory Investigation 6(5):682; Millonig, G. (1961), J. Biophys. Biochem. Cytol. 11:736; and Luft, J. H. (1961), J. Biophys. Biochem. Cyto. 9:409 (Ernest F. Fullam, Inc., Epox 812 Embedding Kit now substituted for Epox 812).

Results: This assay provides electron micrographs of cells exposed to the emulsions which are interpreted by an ultrastructural pathologist, and characterizes the mode and extent of droplet uptake by the cells to determine if the emulsion formulation has the desired membrane penetration features.

The following demonstrate that the direct penetration of the cell surface membrane and the penetration of intracellular membranes (i.e., nuclear membrane) can be achieved by different formulations of the present invention. This type of membrane penetration is unique and is not what has been demonstrated with other nucleic acid transfection vehicles.

Composition No. 1:

An emulsion was prepared from the following components:

1. Mineral oil (light viscosity)=9.5 wt %

2. Solutol HS-15=19.0 wt %

3. Benzyl Alcohol=5.2 wt %

4. NANOPURE™ water=66.2 wt %

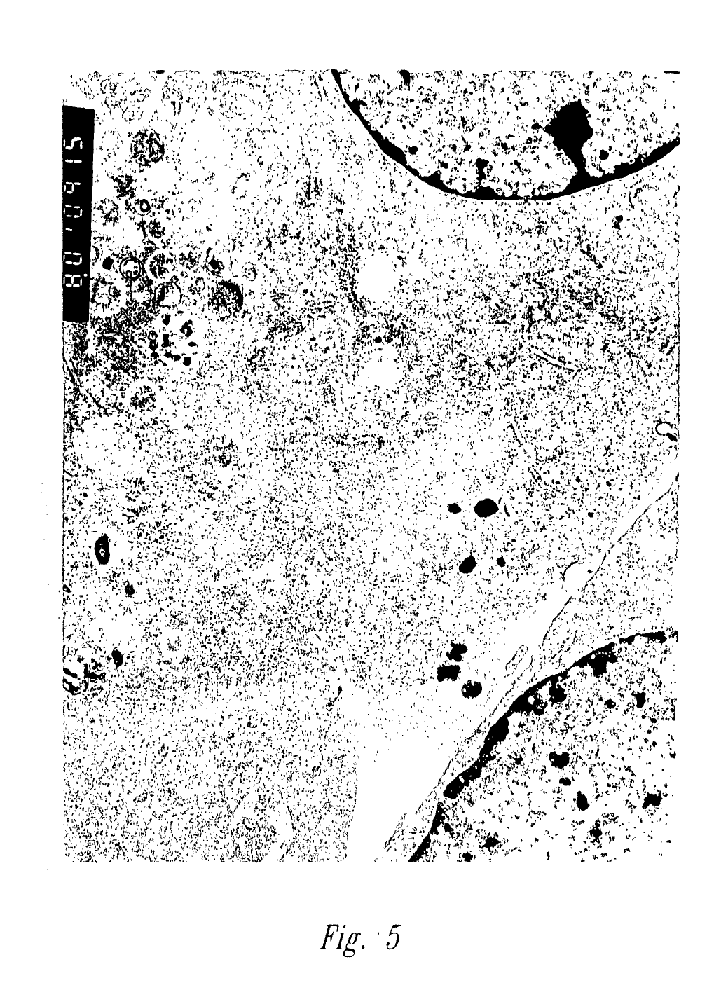

FIG. 5—Electron micrograph 10915: This is an electron micrograph of explanted nonhuman primate (baboon) artery smooth muscle cells incubated with a 1:20 dilution of the emulsion in media for 2 hr, washed and followed by a 24 hr recovery incubation period. Emulsion droplets can be seen between the cells, penetrating the cell membrane directly by microporulation (without a endocytic or phagocytic vesicular membrane) and free within the cytoplasm (8,000×).

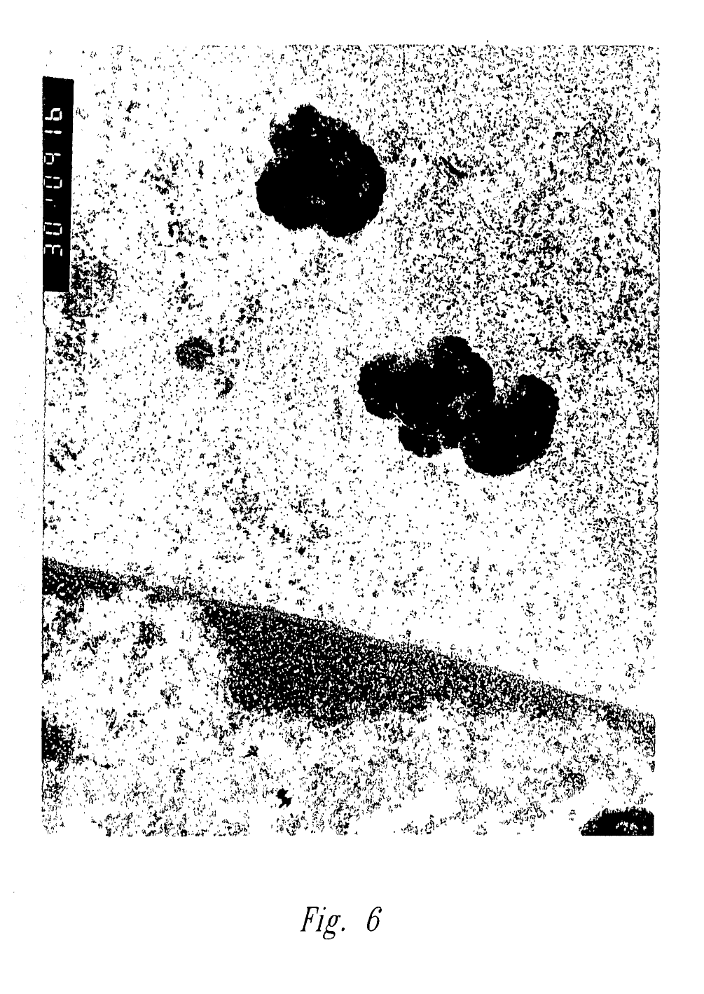

FIG. 6—Electron micrograph 10916: This electron micrograph is from the same tissue culture sample above and demonstrates two clusters of emulsion droplets in the cytoplasm and one emulsion droplet in the nucleus (lower left corner) (30,000×).

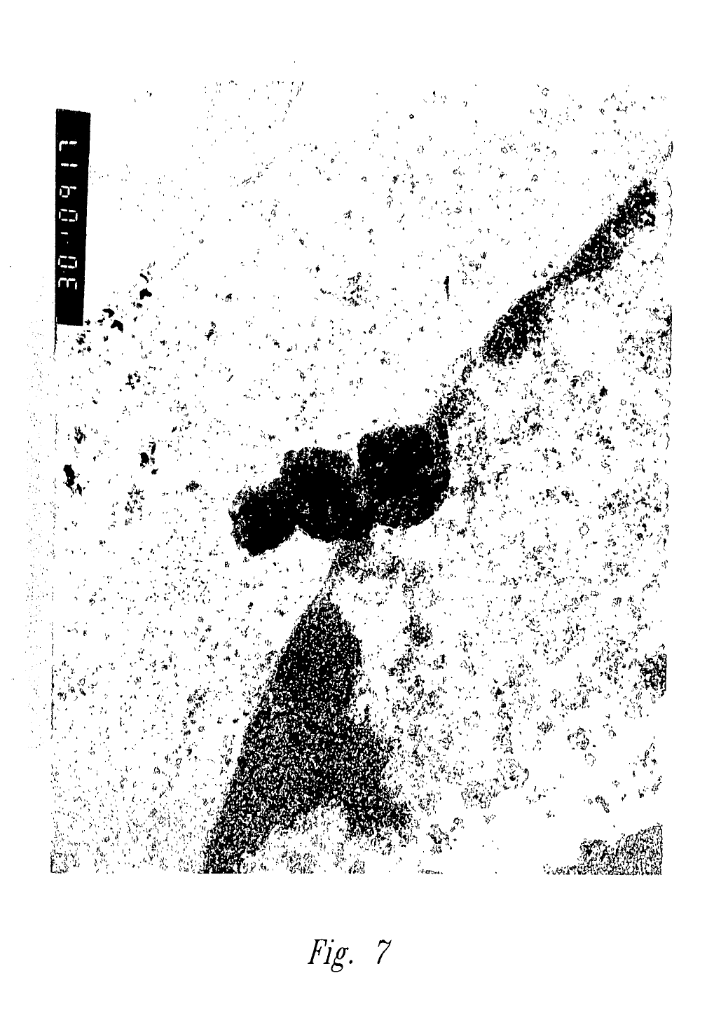

FIG. 7—Electron micrograph 10917: Three emulsion droplets are seen penetrating the nuclear membrane in this smooth muscle cell (30,000×).

Composition No. 2

An emulsion was prepared from the following components:

1. Caprol=5.21 wt %

2. Squalane=13.65 wt %

3. Croval=11.27 wt %

4. NANOPURE™ water=69.86 wt %

5. H³Cytochalasin B=0.003 wt % (0.21 uCi/gm)

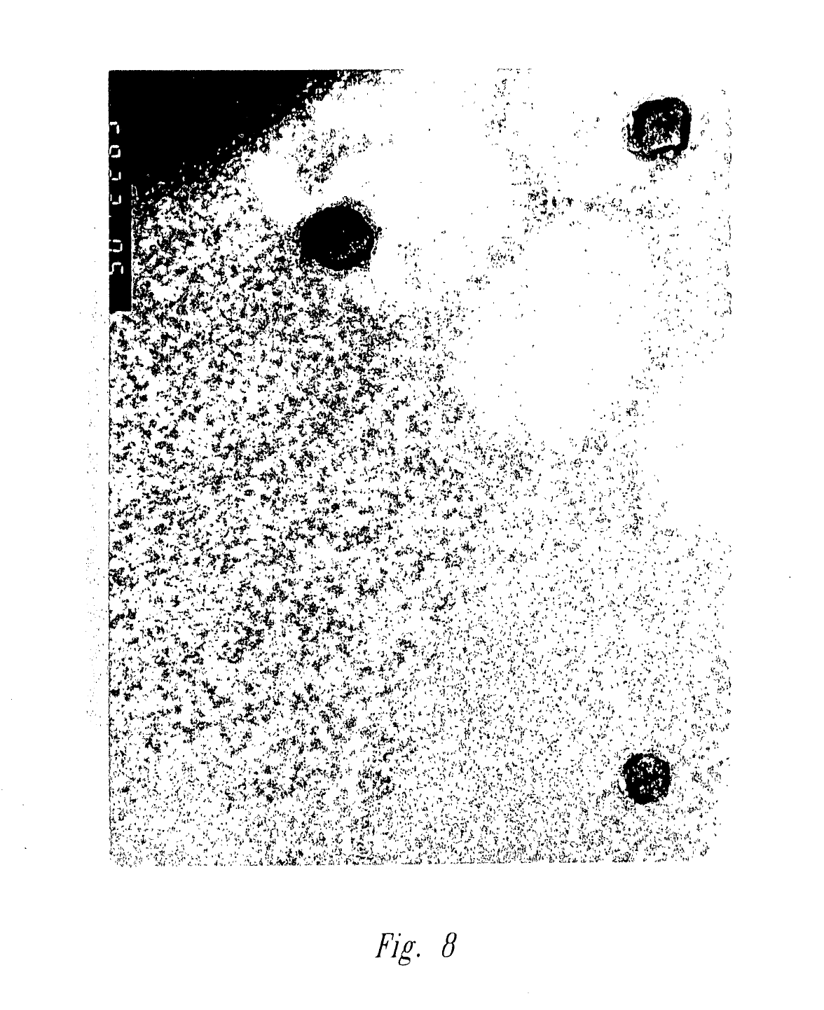

FIG. 8—Electron micrograph 12265: This electron micrograph is of explanted non-human primate (baboon) artery smooth muscle cells incubated with a 1:20 dilution of the emulsion in media for 2 hr, washed and followed by a 24 hr recovery incubation period. Three emulsion droplets are seen in the cytoplasm of the cell (50,000×).

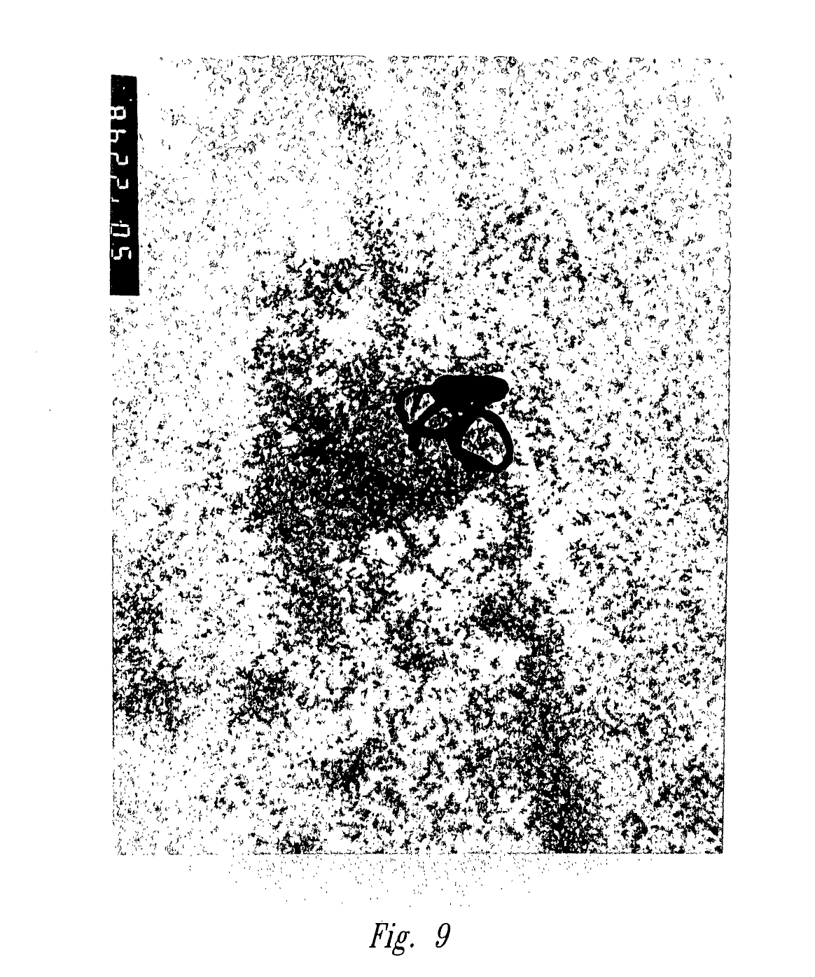

FIG. 9—Electron micrograph 12248: This is an electronmicrographic autoradiography plate of a smooth muscle cell. An emulsion droplet can be seen penetrating the nuclear membrane, and the looped tracts of exposed emulsion outlines the emission tract from the radioactive labeled drug (Cytochalasin B). This plate demonstrates that the emulsion droplets can carry a drug through the cell membrane and nuclear membrane for delivery to the nucleus (50,000×).

Composition No. 3

An emulsion was prepared from the following components:

1. Caprol=7.86 wt %

2. Corn oil=14.62 wt %

3. Cremophor=18.52 wt %

4. NANOPURE™ water=58.99 wt %

5. Coumarin 6=0.008 wt %

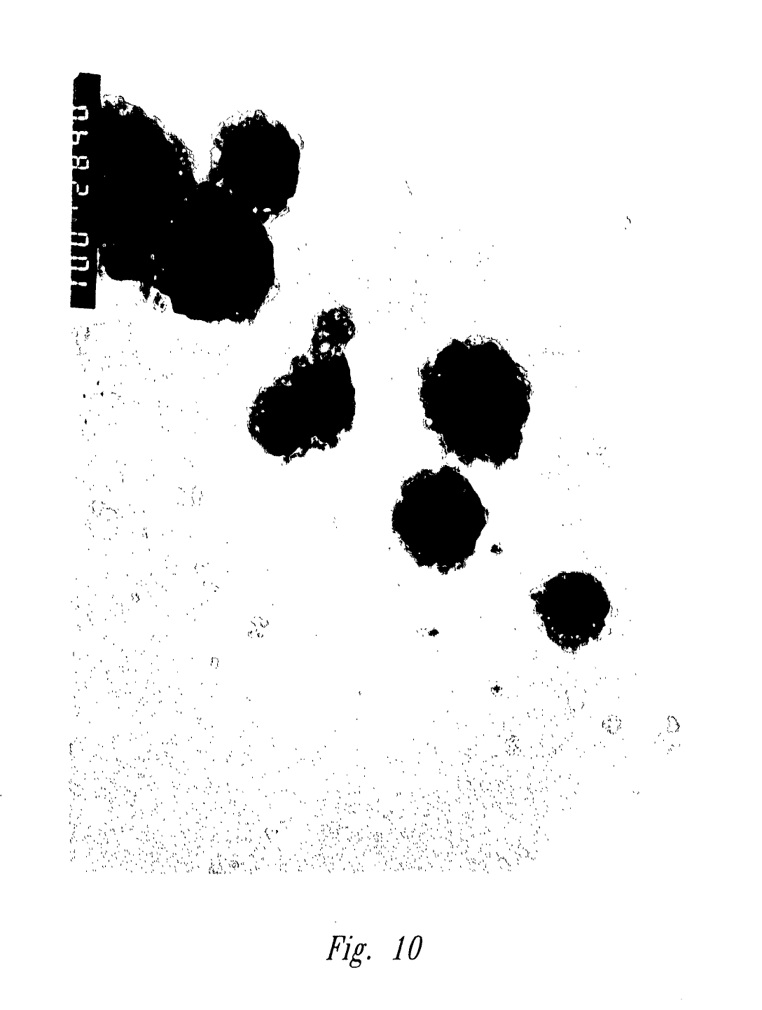

FIG. 10—Electron micrograph 12840: This electron micrograph is of free emulsion droplets from the tissue culture solution, no cells present. The plate demonstrates the small uniform structure of the emulsion droplets (100,000×).

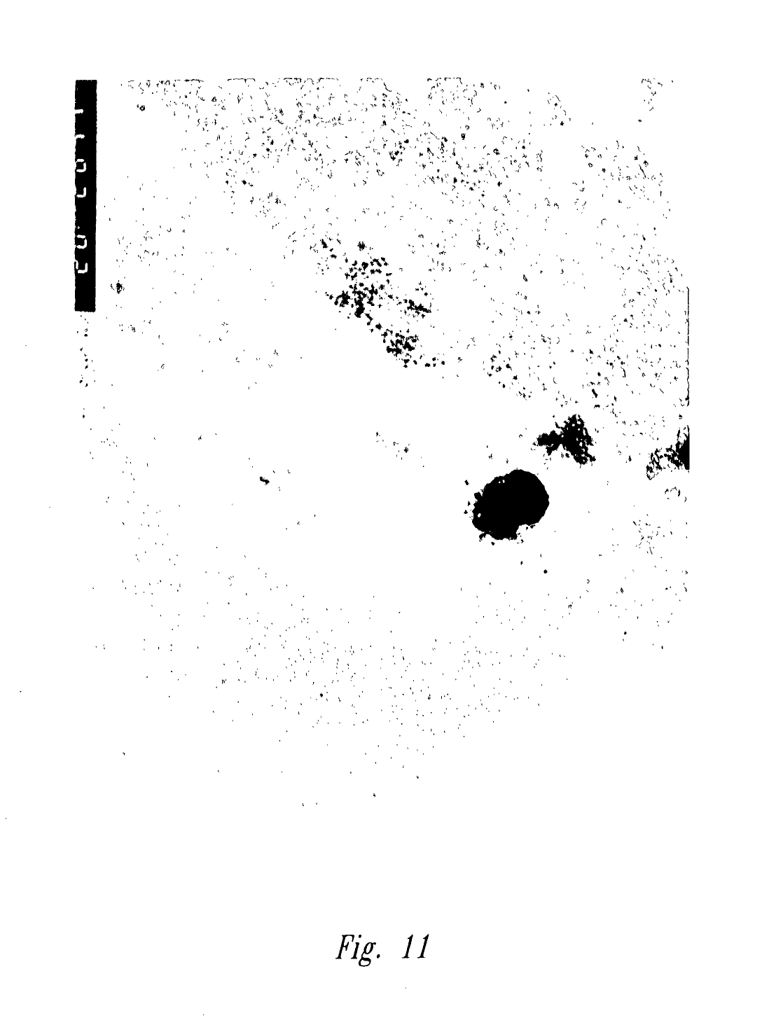

FIG. 11—Electron micrograph 12841: This electron micrograph is of explanted non-human primate (baboon) artery smooth muscle cells incubated with a 1:20 dilution of the emulsion in media for 2 hr, washed and followed by a 24 hr recovery incubation period. Three emulsion droplets are present; one is penetrating the cell membrane directly with no evidence of endocytosis or phagocytosis, the other two have entered the cytoplasm and are in the process of penetrating through the nuclear membrane (20,000×).

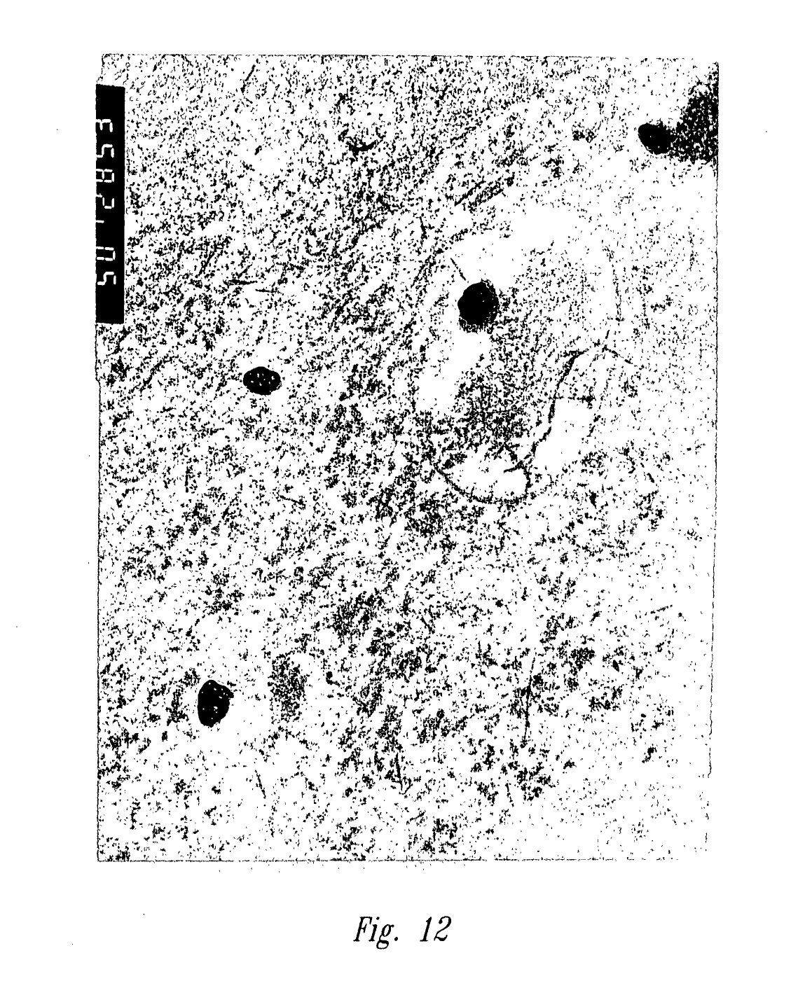

FIG. 12—Electron micrograph 12853: This electron micrograph is of explanted non-human primate (baboon) artery smooth muscle cells incubated the emulsion, which was diluted 1:20 in media, for 30 min and then immediately fixed and processed for evaluation. Multiple emulsion droplets are present in the cytoplasm and one droplet is within a phagocytic vacuole.

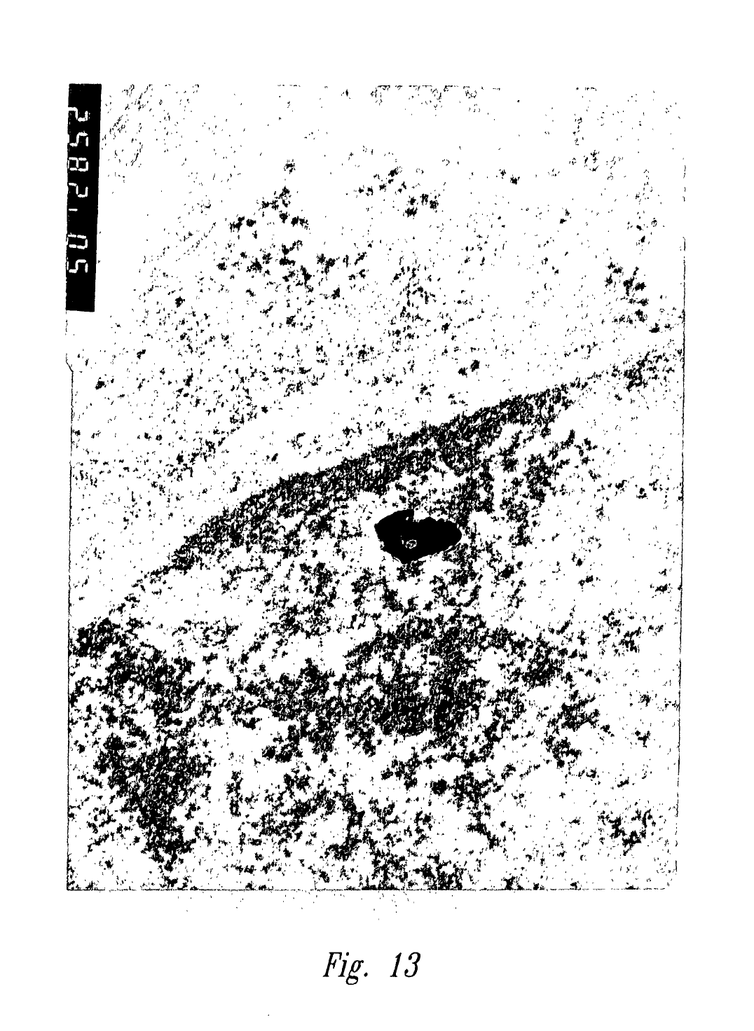

FIG. 13—Electron micrograph 12852: This electron micrograph is of explanted non-human primate (baboon) artery smooth muscle cells incubated with the emulsion, which was diluted 1:20 in media, for 15 min and then immediately fixed and processed for evaluation. An emulsion droplet is present in the nucleus (50,000×).

### Example 15

GENE TRANSFER INTO CHINESE HAMSTER OVARY CELLS

Various microemulsion formulations containing LABRASOL™, pluoral isosterate, and isostearyl isostearate (Gattefossé), as well as the lacZ expression vector pCMVβ (Clonetech, Palo Alto, Calif.), were transfected into Chinese Hamster Ovary (CHO) cells, according to the following procedures.

Preparation of Microemulsions

1. J038-077-05-A, J038-077-50-B, J038-077-100-C: All of the components of the microemulsion were combined in the amounts described in Table 1 below in a glass test tube at room temperature. The components of the microemulsion were combined in no particular order. The mixture was vortexed and placed in a water bath at 40° C. for about 10 minutes. The mixture was removed and vortexed to result in a yellow, transparent microemulsion. Next, three different quantities of a 777 μg/ml aqueous stock solution containing pCMVβ were lyophilized in sterile 1.2mL microfuge tubes at 37° C. for approximately 1-2 hours to yield 5 μg, 50 μg and 100 μg of pCMVβ. The tubes were labeled J038-077-05, J038-077-50, and J038-077-100, respectively. To each tube containing lypophilzed pCMVβ was added 0.5 g of formulation J038-077. The samples were placed at 53° C. bath for 20 minutes and then vortexed. The tubes were replaced in the 53° C. bath for about two hours. The tubes were then removed and allowed to cool to room temperature. The microemulsion samples containing pCMVβ were used immediately in a transfection assay.

TABLE I
MICROEMULSION COMPOSITION COMPONENTS

| Component | J038-077 (wt %) |
|---|---|
| LABRASOL ™ | 32.93 |
| Plurol Isostearate | 13.47 |
| Isostearyl Isostearate | 11.92 |
| USP sterile water | 41.68 |
| Total | 100.00 |

2. J038-096-02 (D), J038-096-03 (E), J038-096-04 (F): On a weight percent basis, 37.37% LABRASOL™, 14.87% Plurol Isostearate and 47.75% USP sterile water were combined in a clear polystyrene vial and labeled J038-096-01. J038-096-01 was vortexed, placed in a 53° C. bath for 1 hour, and then allowed to cool to room temperature. J038-096-01 was yellow and transparent. Next, three different quantities of a 777 μg/ml aqueous stock solution containing pCMVβ were lyophilized in sterile 1.2 mL microfuge tubes at 37° C. for approximately 1-2 hours to yield 5 μg, 50 μg and 100 μg of pCMVβ. The tubes were labeled J038-096-02, J038-096-03, and J038-096-04, respectively. Following lyophilization, a quantity of isostearyl isostearate was added to each of the tubes and the samples were vortexed to disperse the DNA in the oil. The samples were then placed in a 53° C. bath for 1 hour and 15 minutes, with intermittent vortexing. A quantity of J038-096-01 was then added to each tube such that the contents of each tube resulted in the final composition shown in Table 2 below. The samples were vortexed and placed in a 53° C. bath overnight. The samples were vortexed upon removal from the water bath. All three samples were yellow and transparent and appeared to be monophasic. No change occurred on cooling to room temperature.

TABLE 2
MICROEMULSION COMPOSITIONS
WITH DIFFERENT AMOUNTS OF pCMVβ

| | J038-096-02-D | J038-096-03-E | J038-096-04-F |
|---|---|---|---|
| Component | (wt %) | (wt %) | (wt %) |
| LABRASOL ™ | 32.85 | 32.68 | 32.47 |
| Plural isostearate | 13.08 | 13.01 | 12.92 |
| Isostearyl isostearate | 12.09 | 12.54 | 13.12 |
| USP sterile Water | 41.98 | 41.77 | 41.49 |
| Total | 100.00 | 100.00 | 100.00 |

3. J038-124-03 (G): The components of the microemulsion were combined in a clear polystyrene vial in no particular order. Usually the component that was the most difficult to weight out was placed in the vial first. The components were combined in the amounts shown in Table 3 below. After combining the components, the mixture was vortexed and placed in a 53° C. bath for about 2 hours. The mixture was removed and vortexed several times. A yellow, transparent microemulsion resulted. To 2.5 μg of the DIG-lacZ containing pCMVβ vector were added 0.51 g of J038-124-03 in a sterile 2-mL screw calp vial. The sample was vortexed and warmed at 53° C. for about 25 minutes. The samples were removed from the 53° C. bath and allowed to cool to room temperature.

TABLE 3
MICROEMULSION COMPOSITION CONTAINING pCMVβ

| | J038-124-03 |
|---|---|
| Component | (wt %) |
| LABRASOL ™ | 32.76 |
| Plurol Isostearate | 12.88 |
| Isostearyl Isostearate | 12.15 |
| USP sterile water | 42.21 |
| Total | 100.00 |

Transfection of CHO cells with DNA containing Microemulsions: CHO cells are seeded in sterile 96-well plates and incubated overnight at 37° C., 5% CO₂ in a humidified atmosphere. Cells are seeded at 4×10⁴ to 6×10⁴ cells per ml in DMEM with 10% FBS (fetal bovine serum). On the day of transfection, the microemulsion samples are diluted in serum-free DMEM starting at 1/10 in sterile 6 ml tubes. 100 μl per well of test solution is added in duplicate to the CHO cells. The cells are incubated for 1 to 3 hours or overnight at 37° C., 5% CO₂ in a humidified atmosphere. The solution is aspirated in a biosafety cabinet and fresh DMEM with 10% FBS is added. The plates are returned to the incubator for an additional 3-5 days. Following incubation the cells are washed and fixed for histochemical staining and β-galactosidase activity.

Staining for β-galactosidase Activity in CHO cells: The CHO cells in the 96 well plates are placed in a biosafety cabinet and the microemulsion solution is aspirated. The cells are washed with HBSS (Hanks balanced salt solution) and fixed for 10 minutes in a solution containing 2% (v/v) formaldehyde and 0.2% (v/v) glutaraldehyde in PBS (phosphate buffered saline). The cells are washed again in HBSS. Immediately before use, an X-gal (5-bromo-4-chloro-3-indoly-β-D-galactoside, GibcoBRL) solution is made containing: 1 mg/ml X-gal, 2 mM MgCl₂, 5 mM potassium ferrocyanide, 5 mM potassium ferricyanide in PBS. 100 ul/well of X-gal solution is added and the cells are incubated for 1-6 hours at 37° C. To stop the reaction, the X-gal solution is aspirated and the plates are stored in PBS at 4° C. sealed. Cells that turn blue were recorded as positive for β-galactosidase activity (i.e., the lacZ gene was being expressed), while cells that did not develop color were recorded as negative for β-galactosidase activity.

TABLE 4
CHO CELL TRANSFECTION WITH LACZ.

| Formulation | β-galactosidase Activity |
|---|---|
| J038-077-05-A | + |
| J038-077-50-B | + |
| J038-077-100-C | + |
| J038-096-02-D | + |
| J038-096-03-E | − |
| J038-096-04-F | + |
| J038-124-03-G | + |

(+) = β-galactosidase positive cells 3-days after transfection.
(−) = β-galactosidase negative cells 3-days after transfection.

### Example 16

MICROEMULSION DROPLET SIZE ANALYSIS

The uniformity of the droplet size and the stability of the droplets following dilution of the microemulsion liquid crystal phase is important in characterizing the formulations designed to carry different constructs or compounds. In evaluating the various microemulsion formulations, two methods have been employed to measure the droplet size. The most rapid and economical method is the use of a laser light diffraction particle analyzer, which uses deconvolution computer software to both count and size the droplets. The other method, electron microscopy, is more expensive and time consuming, but provides morphology data that is lacking in laser light diffraction analyses.

Table 5 summarizes the size data for six samples examined by laser light diffraction particle analyzer to evaluate batch-to-batch size reproducibility. The six microemulsion samples are based on the following composition: Cytochalasin B (0.003 wt %), Caprol 10G40 (5.23 wt %), Squalane (13.63 wt %), Crovol A-70 (11.17 wt %), and NANOPURE™ water (69.97 wt %). The samples are examined at the time of dilution (time 0) and 6 hours after dilution to evaluate droplet stability. The dilution used simulates the dilution that occurs following in vivo administration to test animals and humans.

TABLE 5
RESULTS OF MICROEMULSION DROPLET SIZE ANALYSIS

| | Mean | Mean | Mean | Mean |
|---|---|---|---|---|
| | Size, nm | Size, nm | Size, nm | Size, nm |
| | (1:20 | (1:20 | (1:200 | (1:200 |
| Sample | dilution) | dilution) | dilution) | dilution) |
| Number | @ 0 hrs. | @ 6 hrs. | @ 0 hrs. | @ 6 hrs. |
| I108-102-01a | 27.1 | 28.4 | 30.0 | 31.5 |
| I108-102-01b | 29.5 | 29.9 | 32.1 | 33.8 |
| I108-102-01c | 29.0 | 28.6 | 28.7 | 40.3 |
| I108-102-01d | 28.6 | 29.1 | 30.8 | 29.1 |
| I108-102-01e | 28.8 | 29.8 | 31.2 | 44.5 |
| I108-102-01f | 29.5 | 29.4 | 32.7 | 40.6 |
| Mean Size | 28.8 ± 0.6 | 29.2 ± 0.5 | 30.9 ± 1.1 | 36.6 ± 5.2 |

The data shows the uniformity of the droplet size and the stability of this formulation at the 1:20 dilution for up to 6 hours. The initial sizes of the droplets in the 1:200 dilution are consistent; however, there is early degradation and droplet enlargement by 6 hours. Based on electron microscopic studies of tissue culture cell penetration by microemulsions, the 6 hour stability is sufficient for the droplets to penetrate cells both in vitro and in vivo.

For electron microscopic (EM) sizing, dilutions of the microemulsion are placed in two chamber tissue culture slides as a thin film, dried and processed as described in Example 14. The size of the droplets are more variable because the 80-100 angstrom thick sections are taken at different levels through the spherical droplets with each section having a different diameter. The largest droplets in the EM images represent the sectioning plane through the spherical equator, and the smaller droplets represent sectioning planes nearer the poles of the sphere. The diameter of the larger droplets should correspond to the size determined by the laser light diffraction particle analyzer. In reality, the EM droplet diameters appear larger due to the heat of the electron beam melting and spreading the droplets variable amounts. The EM sizing data of microemulsion composition no. 3 (see Example 14) demonstrates the variability of the cross-sections of the droplet spheres (Table 6).

TABLE 6
EM MEASURED DROPLET SIZE DATA

| Sample Number | Mean Size, nm |
|---|---|
| 1 | 375 |
| 2 | 320 |
| 3 | 250 |
| 4 | 300 |
| 5 | 275 |
| 6 | 400 |
| 7 | 310 |
| Average | 318.57 ± 53 |

### Example 17

MICROEMULSIONS CROSS THE BLOOD-BRAIN BARRIER

Cytochalasin B microemulsion H906-097-01 (cytochalasin B, 0.004%; caprol 10G40, 5%; squalene, 9.5%; crovol A-70, 9.5%; and NANOPURE™ water, 76%) was diluted 1:20 in saline. Six adult male rats were each injected intravenously with 5 ml of the diluted emulsion. Then various organs (such as brain, liver, spleen, lung, and mesenteric lymph nodes) were harvested 4, 7, and 21 days after injection to examine the histopathology.

The following histopathogy results from brain tissue indicate the cytochalasin B microemulsion has the unique ability to cross the blood-brain barrier. The cerebral vascular endothelial cells were plump and there were reactive glial cells surrounding a cerebral blood vessel. The perivascular aggregation of glial cells indicated that the glial cells were unable to migrate. In other words, the glial cell aggregation could occur only if the microemulsion had crossed the blood-brain barrier and if the cytochalasin B had entered the cells to block their ability to migrate away from the preivascular region.

### Example 18

PLASMID STABILITY IN MICROEMULSIONS

The lacZ-containing vector, pCMVβ, was incorporated into a microemulsion composition comprising 32.93 wt % LABRASOL™, 13.47 wt. % Plurol isostcarate, 11.92% isostearyl isostearate and 41.68% USP sterile water wherein 5, 50 or 100 ug of plasmid was present. The plasmid/microemulsion complexes were stored at 4° C. for 8 and 10 weeks, then the DNA was analyzed by electrophoresis on 0.8% agarose gels. The plasmid showed no apparent loss of stability as a result of residing within the microemulsion composition. Furthermore, the microemulsions were also tested in transfection studies as described in Example 15, with results comparable to those shown in Example 15.

### Example 19

GENE TARGETING IN VIVO

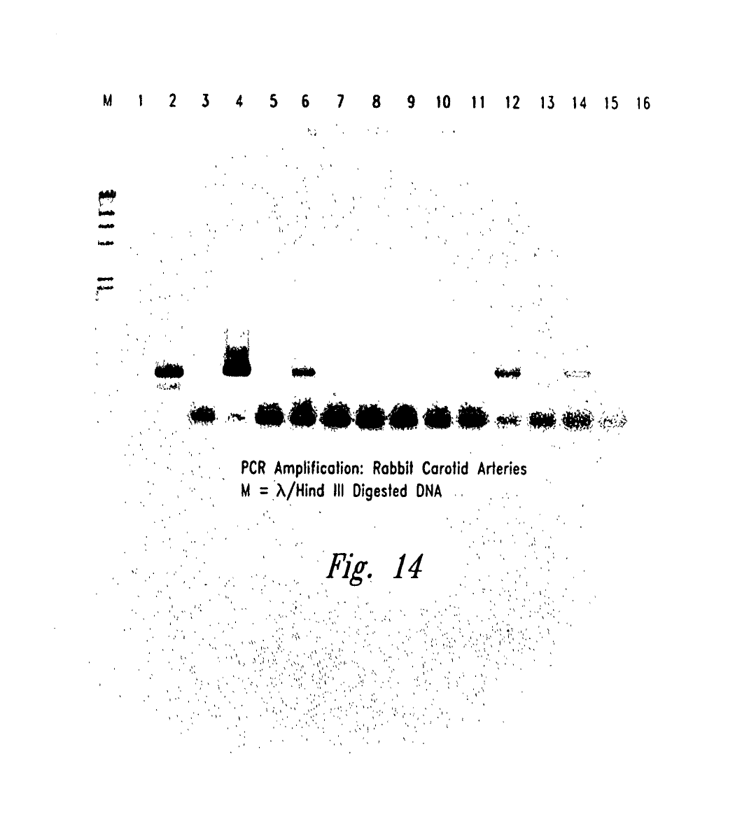

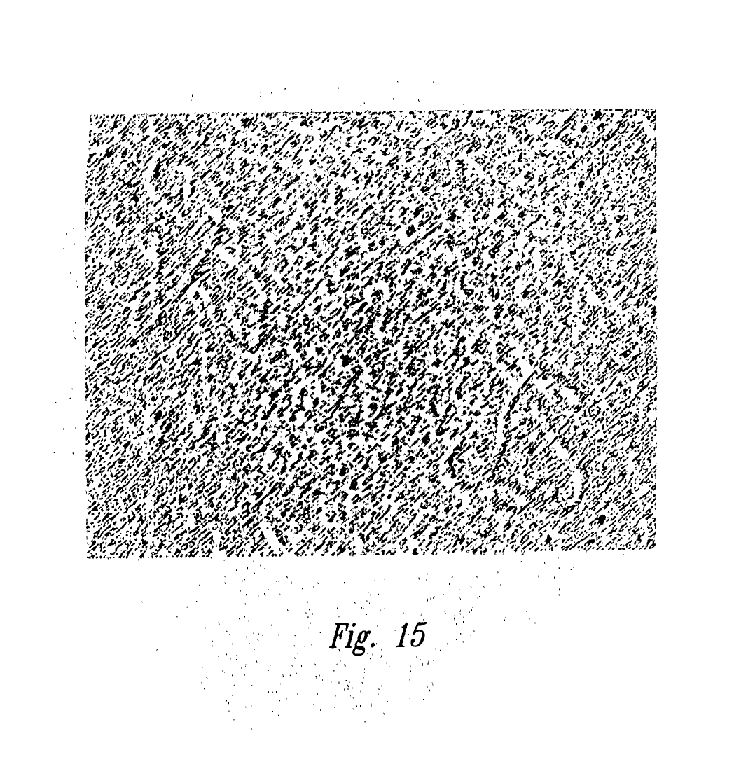

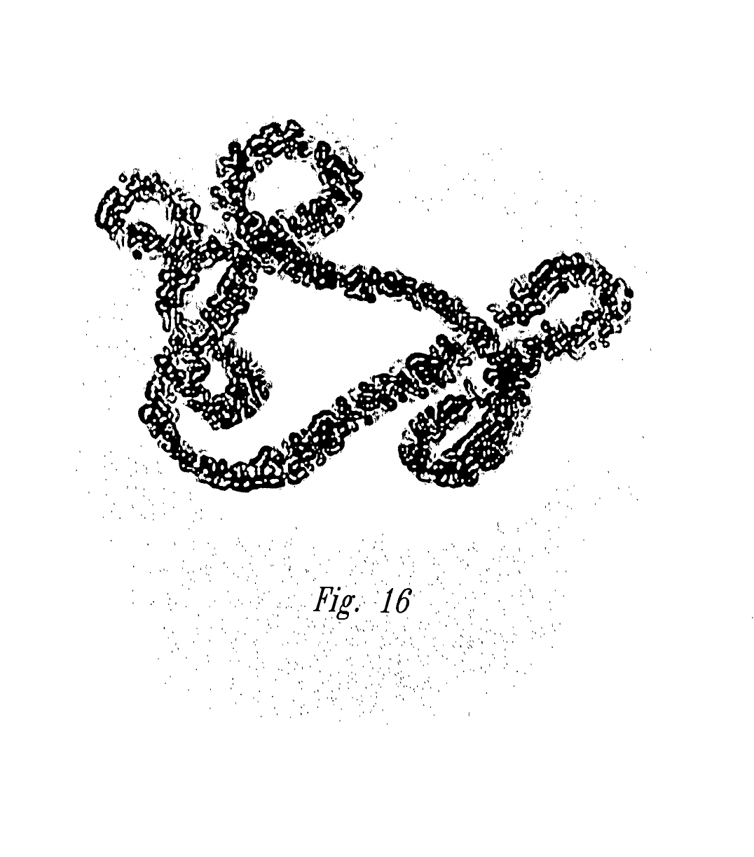

Rabbit carotid arteries were infused with the pbetagal microemulsion composition of Example 18. Sections from either the left (infused) or right (control) artery were harvested and immediately frozen on dry ice on days 4, 7, 9, 12 , and 14 days after infusion. Using standard procedures described in the art, total genomic DNA was extracted and subjected to the polymerase chain reaction (PCR). The PCR primers used were designed to generate a 520 base pair fragment that is within the coding region of lacZ. The resulting amplification products were visualized by electrophoresis through a 1% agarose gel (FIG. 14). The lacZ gene was present in the arterial tissue that had been infused with the microemulsion and not in the control arterial tissue. Moreover, the 520 base pair amplification product was confirmed to be lacZ by using dideoxy sequencing as described in the art. Additionally, in situ PCR was performed (see the Detailed Description) to confirm that pbetagal was subcellularly localized within the carotid artery.

All publications and patent applications mentioned in this specification are herein incorporated by reference to the same extent as if each individual publication or patent application was specifically and individually incorporated by reference.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed:

1. A composition for transporting a nucleic acid across a biological membrane, the composition comprising a nucleic acid, an oil, an oil-immiscible compound and a noncationic surface active agent, the composition being able to transport the nucleic acid across the membrane.

2. The composition of claim 1, wherein the composition contains essentially no cationic lipid.

3. The composition of claim 1, wherein the composition comprises two noncationic surface active agents.

4. The composition of claim 1, wherein the nucleic acid comprises adenine, guanine, cytosine, thymine or uracil, or a polymer containing a plurality of residues thereof.

5. The composition of claim 1, wherein the nucleic acid is a gene.

6. The composition of claim 1, wherein the nucleic acid is a hydrophobically-modified nucleic acid.

7. The composition of claim 6, wherein the hydrophobically-modified nucleic acid is a hydrophobically-modified nucleic acid vaccine.

8. The composition of claim 7, wherein the hydrophobically-modified nucleic acid contains a hydrophobic moiety covalently bonded to the nucleic acid, and the hydrophobic moiety is selected from steroids and digoxigenin.

9. The composition of claim 1, wherein the nucleic acid is a nucleic acid vaccine.

10. The composition of claim 9, wherein the nucleic acid vaccine is a DNA vaccine.

11. The composition of claim 9, wherein the nucleic acid vaccine is capable of inducing at least one of a cytotoxic T-lymphocyte (CTL) response, a T-helper response, and a neutralizing antibody response in a subject to whom the composition is administered.

12. The composition of claim 9, wherein the nucleic acid vaccine is capable of potentiating at least one of a pre-existing CTL response to an antigen, a pre-existing T-helper response to an antigen, and a pre-existing neutralizing antibody response to an antigen, in a subject to whom the composition is administered.

13. The composition of claim 1, wherein the oil comprises a vegetable oil.

14. The composition of claim 13, wherein the vegetable oil comprises castor oil, coconut oil, corn oil, ethereal oil, olive oil, palm oil, peanut oil, rape oil, sesame oil, or a combination thereof.

15. The composition of claim 13, wherein the oil is miscible with water to an extent of less than about 0.1 g oil per about 100 g water at 25° C.

16. The composition of claim 1, wherein the oil-immiscible compound comprises an aqueous solution.

17. The composition of claim 16, wherein the oil-immiscible compound is soluble in corn oil to an extent of less than about 0.1 g oil-immiscible compound per about 100 g corn oil at 25° C.

18. The composition of claim 1, wherein the noncationic surface active agent comprises a nonionic surface active agent.

19. The composition of claim 18, wherein polyethylene glycol is incorporated into the noncationic surface active agent.

20. The composition of claim 18, wherein polyglycerol is incorporated into the noncationic surface active agent.

21. The composition of claim 1, wherein the noncationic surface active agent comprises an anionic surface active agent.

22. The composition of claim 20, wherein polyethylene glycol is incorporated into the noncationic surface active agent.

23. The composition of claim 20, wherein polyglycerol is incorporated into the noncationic surface active agent.

24. The composition of claim 1 comprising first and second noncationic surface active agents of non-identical structures.

25. The composition of claim 24, wherein the first noncationic surface active agent has a hydrophilic-lipophillic balance number (HLB) of 8 or less and the second noncationic surface active agent has a HLB of 10 or greater.

26. The composition of claim 24, wherein the first and second noncationic surface active agents are both nonionic.

27. The composition of claim 1 having no effective amount of cationic surfactant.

28. The composition of claim 1 having no cationic surfactant.

29. The composition of claim 1 comprising a nonionic surfactant which is primarily dissolved in an oil phase, and a nonionic surfactant which is primarily dissolved in an oil-immiscible phase.

30. The composition of claim 1 in the form of an emulsion.

31. The composition of claim 1 in the form of a microemulsion.

32. The composition of claim 1 comprising droplets, wherein greater than about 90% of the droplets have a diameter within the range of about 0.1 nm to about 500 nm.

33. The composition of claim 1, wherein the composition comprises a water-in-oil (W/O) emulsion or microemulsion.

34. The composition of claim 1, wherein the oil-immiscible compound comprises an aqueous continuous phase.

35. The composition of claim 1 which is self-emulsifying.

36. The composition of claim 1, wherein the oil-immiscible compound comprises an oil continuous phase.

37. The composition of claim 1 which is an oil-in-water (O/W) emulsion or microemulsion.

38. The composition of claim 1 which is bicontinuous.

39. The composition of claim 1 which is biocompatible.

40. The composition of claim 1, wherein the biological membrane is a lipid membrane of an animal cell.

41. The composition of claim 1, wherein the cell comprises a mammalian cell, an avian cell or a fish cell.

42. The composition of claim 1, wherein the composition comprises a hydrophobically-modified nucleic acid and
(i) an oil, an oil-immiscible compound, a noncationic surface active agent, and essentially no cationic lipid; and/or
(ii) an oil, an oil-immiscible compound, and two noncationic surface active agents.

43. A method of forming the composition of claim 1, comprising mixing a nucleic acid, an oil component, an oil-immiscible component, and a noncationic surface active agent.

44. The method of claim 43, wherein the noncationic surfactant comprises at least two noncationic surfactants.

45. The method of claim 43, wherein in the mixing step, essentially no cationic lipid is used.

46. The method of claim 43, wherein the nucleic acid comprises a hydrophobically-modified nucleic acid.

47. The method of claim 43, wherein the nucleic acid is a nucleic acid vaccine.

48. The method of claim 43, wherein the nucleic acid vaccine is a DNA vaccine.

49. The method of claim 43, wherein the nucleic acid is hydrophobically-modified nucleic acid, and the hydrophobically-modified nucleic acid is mixed with
(i) an oil, an oil-immiscible compound, a noncationic surface active agent, and essentially no cationic lipid; and/or
(ii) an oil, an oil-immiscible compound, and two noncationic surface active agents.

50. A method of delivering a nucleic acid into a cell, comprising contacting the cell with the composition of claim 1, whereby the nucleic acid is delivered into the cell.

51. The method of claim 50, wherein the composition comprises at least one of the following compositions:
(a) a composition comprising a nucleic acid, an oil component, an oil-immiscible compound, and a noncationic surface active agent and essentially no cationic lipid;
(b) a composition comprising a nucleic acid, an oil component, an oil-immiscible compound, and two noncationic surface active agents; and/or
(c) a composition comprising a hydrophobically-modified nucleic acid, an oil component, an oil-immiscible compound and at least one surface active agent,
whereby the nucleic acid is delivered into the cell.

52. The method of claim 50, wherein the cells are contacted in vitro with the composition for a sufficient period of time for delivery of the nucleic acid into one or more cells to occur, and the thus contacted cells are then introduced into a subject.

53. The method of claim 50, wherein the contacting takes place for a time of about 5 minutes to about 48 hours and at a temperature of about 25° C. to about 45° C.

54. The method of claim 50, wherein the cells are contacted in vivo with the composition for a sufficient period of time for delivery of the nucleic acid into one or more cells of a subject.

55. The method of claim 54, wherein the contacting is accomplished through parenteral administration of the composition to the subject.

56. The method of claim 54, wherein the cell is a smooth muscle cell.

57. The method of claim 54, wherein the contacting results in integration of a functional copy of a gene into a chromosome of a cell of the subject.

58. The method of claim 50, wherein the contacting introduces a fragment of a gene into the nucleus of the cell.

59. The method of claim 50, wherein the nucleic acid is a nucleic acid vaccine.

60. The method of claim 59, wherein the nucleic acid vaccine is a DNA vaccine.

61. The method of claim 59, wherein the nucleic acid vaccine induces an immune response in a subject.

62. The method of claim 59, wherein the nucleic acid vaccine induces at least one of a CTL response, a T-helper response and a neutralizing antibody response in a subject.

63. The method of claim 59, wherein the nucleic acid vaccine potentiates at least one of a pre-existing CTL response to an antigen, a pre-existing T-helper response to an antigen, and a pre-existing neutralizing antibody response to an antigen in a subject.

64. The method of claim 50, wherein the cell is an animal cell.
